(19) United States
(12) Patent Application Publication
(10) Pub. No.: US 2023/0340606 A1
(43) Pub. Date: Oct. 26, 2023

(54) EXTRACELLULAR REPETITIVE RNA BIOMARKERS OF MUTANT KRAS CANCERS

(71) Applicant: The Regents of the University of California, Oakland, CA (US)

(72) Inventors: Daniel H. KIM, Santa Cruz, CA (US); Roman REGGIARDO, Santa Cruz, CA (US)

(73) Assignee: The Regents of the University of California, Oakland, CA (US)

(21) Appl. No.: 17/941,270

(22) Filed: Sep. 9, 2022

Related U.S. Application Data
(60) Provisional application No. 63/242,247, filed on Sep. 9, 2021.

(51) Int. Cl. C12Q 1/6886

(52) U.S. Cl. CPC C12Q 1/6886; C12Q 2600/158; C12Q 2600/156

(57) ABSTRACT

The disclosure provides methods for detecting a RAS pathway mutation in a subject. The methods include obtaining a biological sample from the subject, isolating nucleic acids from the biological sample, and analyzing the expression level of noncoding RNAs in the nucleic acids in conjunction with a corresponding reference level in a control sample, wherein a differential expression level of the noncoding RNAs compared to the corresponding reference level in the control sample indicates that the subject has a RAS pathway mutation.

### CROSS-REFERENCE TO RELATED APPLICATIONS

This application claims priority to U.S. Provisional Application No. 63/242,247, filed Sep. 9, 2021, the disclosure of which is hereby incorporated by reference in its entirety for all purposes.

### BACKGROUND

Oncogenic KRAS is a potent initiator of tumorigenesis, yet its nascent effects on the noncoding genome are incompletely understood.

### SUMMARY

In one aspect, the disclosure provides methods for detecting a RAS pathway mutations in a subject. The methods include obtaining a biological sample from the subject, isolating nucleic acids from the biological sample, and analyzing the expression level of noncoding RNAs in the nucleic acids in conjunction with a corresponding reference level in a control sample, wherein a differential expression level of the noncoding RNAs compared to the corresponding reference level in the control sample indicates that the subject has a RAS pathway mutation.

In some embodiments, the method further comprises, after analyzing, administering to the subject one or more anticancer agents. In certain embodiments, the anticancer agent is an inhibitor of a K-ras gene. In other embodiments, the anticancer agent is an inhibitor of the gene that is identified to have the differential expression level compared to the corresponding reference level for the gene in the control sample.

In some embodiments, the cancer comprises a KRAS mutation. The KRAS mutation can be in a tissue of the subject, such as lung tissue. In certain embodiments, the cancer is lung cancer, such as lung adenocarcinoma.

In some embodiments, the method comprises analyzing the expression level of a gene involved in the interferon (IFN) alpha or gamma response. In certain embodiments, an increase in the expression level of the gene involved in the IFN alpha or gamma response relative to a corresponding reference level for the gene in the control sample from the control subject indicates that the subject has cancer.

In some embodiments, the method comprises analyzing the expression level of a gene encoding a KRAB zinc-finger (KZNF) protein. In certain embodiments, a decrease in the expression level of the gene encoding the KZNF protein relative to a corresponding reference level for the gene in the control sample from the control subject indicates that the subject has cancer.

In some embodiments, measuring the expression level of the one or more genes comprises performing polymerase chain reaction (PCR), reverse transcriptase polymerase chain reaction (RT-PCR), single-cell RNA-sequencing, microarray analysis, a Northern blot, serial analysis of gene expression (SAGE), immunoassay, hybridization capture, cDNA sequencing, direct RNA sequencing, nanopore sequencing, a CRISPR based technology and/or mass spectrometry. Specifically, when PCR is used to measure the expression level, at least one set of oligonucleotide primers comprising a forward primer and a reverse primer capable of amplifying a polynucleotide sequence of the gene can be used.

In some embodiments, the biological sample is a blood sample, a urine sample, a saliva sample, or a tissue sample (e.g., a blood sample). In some embodiments, the subject suspected of having cancer or in need of treatment is a mammal (e.g., a human).

As used herein, the term “RAS pathway mutation” refers to a genetic mutation in a RAS pathway gene. Optionally, the RAS pathway mutation comprises a mutation in KRAS, NRAS, HRAS, EGFR, NF1, MET or BRAF. Optionally, the mutation is in KRAS. As used herein, the term “KRAS mutation” refers to a genetic mutation in the KRAS gene, which acts as an on-off switch in cell signaling and controls cell proliferation.

As used herein, the term “long noncoding RNA” or “lncRNA” refers to RNA polynucleotides that are not translated into proteins. Long ncRNAs may vary in length from several hundred bases to tens of kilo bases (e.g., at least 100, 200, 300, 400, 500, 600, 700, 800, 900, 1000, 1100, 1200, 1300, 1400, 1500, 1600, 1700, 1800, 1900, or 2000 bases) and may be located separately from protein coding genes, or reside near or within protein coding genes.

As used herein, the term “polynucleotide” refers to an oligonucleotide, or nucleotide, and fragments or portions thereof, and to DNA or RNA of genomic or synthetic origin, which may be single- or double-stranded, and represent the sense or anti-sense strand. A single polynucleotide can be translated into a single polypeptide unless it is noncoding.

As used herein, the terms “peptide” and “polypeptide” are used interchangeably and describe a single polymer in which the monomers are amino acid residues which are joined together through amide bonds. A polypeptide is intended to encompass any amino acid sequence, either naturally occurring, recombinant, or synthetically produced.

As used herein, the term “substantial identity” or “substantially identical,” used in the context of nucleic acids or polypeptides, refers to a sequence that has at least 50% sequence identity with a reference sequence. Alternatively, percent identity can be any integer from 50% to 100%. In some embodiments, a sequence is substantially identical to a reference sequence if the sequence has at least 50%, 55%, 60%, 65%, 70%, 75%, 80%, 85%, 90%, 91%, 92%, 93%, 94%, 95%, 96%, 97%, 98%, or 99% sequence identity to the reference sequence as determined using, e.g., BLAST.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A comparison window includes reference to a segment of any one of the number of contiguous positions, e.g., a segment of at least 10 residues. In some embodiments, the comparison window has from 10 to 600 residues, e.g., about 10 to about 30 residues, about 10 to about 20 residues, about 50 to about 200 residues, or about 100 to about 150 residues, in which a sequence may be compared to a reference sequence of the same number of contiguous positions after the two sequences are optimally aligned.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=-2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference sequence if the smallest sum probability in a comparison of the test amino acid sequence to the reference amino acid sequence is less than about 0.01, more preferably less than about 10⁻⁵, and most preferably less than about 10⁻²⁰.

### BRIEF DESCRIPTION OF THE DRAWINGS

FIGS. 1A-1E: mutant KRAS induces an intrinsic IFN-stimulated gene signature. Volcano plot depicting the differential expression of IFN-stimulated genes in mutant KRAS AALEs (FIG. 1A). Venn diagram highlighting the unique and shared genes across GSEAenriched IFN gene sets (FIG. 1B). Seurat UMAP with k-means clusters representation of single cell RNA sequencing results (FIG. 1C). Violin plots depicting the distribution of averaged gene set expression in cells belonging to each k-means cluster (FIG. 1D). Distributions of normalized counts for DE ISGs across Seurat-identified clusters of single cells (FIG. 1E).

FIGS. 2A-2E: Epigenetic reprogramming of ISGs by mutant KRAS. Relative ATAC-seq peak density (KRAS/CTRL) calculated proximal to the TSS of genes within each respective gene set (FIG. 2A). Annotations of ISG ATAC-seq peaks unique to KRAS AALEs. * indicates 2 unique peaks (FIG. 2B). UCSC Genome Browser tracks of ATAC-seq and RNA-seq alignments in both KRAS and CTRL AALEs for IRF9, OAS2, and IFIT1 (FIG. 2C). Selected enriched motifs from the unique KRAS ATAC-seq peaks assigned to ISGs by Homer (FIG. 2D). Volcano plot of all differentially expressed transcription factors with motifs in unique KRAS ATAC peaks assigned to ISGs by Homer (FIG. 2E).

FIGS. 3A-3E: Mutant KRAS regulates repetitive noncoding RNA expression and editing. Volcano plot of all differentially expressed TE species aggregated from individual loci (FIG. 3A). Violin plots of the distribution of TE family expression across each single cell cluster (FIG. 3B). Violin plots depicting distribution of L1MC4a and AluSx expression across single cells clusters (FIG. 3C). Boxplots of Alu RNA editing from equal subsamples of cells from each single cell cluster (FIG. 3D). Distribution of normalized counts for PKR, MDA5, and RIG-I across Seuratidentified clusters (FIG. 3E).

FIGS. 4A-4E: Broad epigenetic silencing of KRAB zinc-finger genes by mutant KRAS. Volcano plot of all differentially expressed zinc-finger genes with known annotations (FIG. 4A). Relative ATAC-seq peak density (KRAS/CTRL) calculated proximal to the TSS of KZNF genes and annotations of ATAC-seq peaks assigned to KZNFs and unique to CTRL AALEs. * indicates 2 unique peaks (FIG. 4B). UCSC Genome Browser tracks of ATAC-seq and RNA-seq alignments in both KRAS and CTRL AALEs of down-regulated ZNF90, ZNF736, and ZNF682 (FIG. 4C). Selected enriched motifs from the unique KRAS ATAC-seq peaks assigned to KZNFs by Homer (FIG. 4D). Model of epigenomic reprogramming of KZNFs, repetitive noncoding RNAs, and IFN-stimulated genes by mutant KRAS (created with BioRender.com) (FIG. 4E).

FIGS. 5A-5E: Mutant KRAS reprograms the transcriptome. Schematic of experimental workflow (created with BioRender.com) (FIG. 5A). Volcano plot depicting the differential expression of GENCODE genes in mutant KRAS AALEs (FIG. 5B). GSEA normalized enrichment score for differentially expressed genes in mutant KRAS AALEs (FIG. 5C). Volcano plot depicting the differential expression of GENCODE genes in mutant KRAS HA1Es (FIG. 5D). GSEA normalized enrichment score for differentially expressed genes in mutant KRAS HAlEs (FIG. 5E).

FIGS. 6A-6E: Extracellular RNAs released from mutant KRAS AALEs. Ribbon plot of extracellular vesicle size abundance from nanoparticle tracking analysis. Solid line is mean of three measurements and the surrounding filled-in area reflects their standard deviation. Solid points are local maxima with corresponding value labelled adjacently (FIG. 6A). Volcano plots depicting the differential enrichment of lncRNA and proteincoding genes in mutant KRAS AALE extracellular RNAs (FIG. 6B). Correlation between intracellular and extracellular RNAs that are differentially expressed/enriched from mutant KRAS AALEs (FIG. 6C). Boxplot comparing distributions of average counts across conditions in TE clades, significance determined by one-way t-test (FIG. 6D). Boxplot of detected Alu editing in three replicates of RNA-seq performed on extracellular vesicles isolated from media from each condition (FIG. 6E).

FIGS. 7A-7C: Mutant KRAS represses KZNFs in vitro and in vivo. UCSC genome browser tracks of ATAC-seq and RNA-seq alignments in both KRAS and CTRL AALEs of down-regulated ZNF826P, ZNF382, and ZSCAN18 (FIG. 7A). UCSC repeat browser tracks of ChIP-seq peaks for differentially expressed KZNFs over indicated TE consensus sequences (FIG. 7B). Distribution of differentially expressed ZNFs (in mutant KRAS AALEs) in RNA-seq data from GTEx lung tissues and TCGA lung adenocarcinomas by KRAS mutation status (FIG. 7C).

### DETAILED DESCRIPTION

### I. Introduction

Most of the human genome is noncoding and transcribed into RNA (1, 2), but how the noncoding transcriptome contributes to cancer formation is poorly understood. About half of the human genome is comprised of transposable elements (TE) (3), whose expression patterns are often altered in cancer (4) . Additionally, TEs contribute substantially to the noncoding transcriptome and are present in the exonic sequences of thousands of long noncoding RNAs (lncRNAs) and other classes of regulatory RNAs (5). Noncoding RNA networks become disrupted in cancer (6, 7) and epigenetic reprogramming, where early activation of RAS signaling leads to coordinate activation of noncoding RNAs in single cells (8). While RAS genes are among the most frequently mutated oncogenes in cancer (9), the extent to which RAS regulates the noncoding transcriptome during cellular transformation remains unknown.

To determine the landscape of noncoding RNAs affected by oncogenic RAS signaling, we performed RNA sequencing (RNA-seq) on human lung epithelial cells (AALE) that undergo malignant transformation upon introduction of mutant KRAS (10). The transcriptomes of AALE cells transduced with control vector were compared to AALEs that were transformed by mutant KRAS and analyzed the distribution of differentially expressed transcripts across the genome.

### II. Transcriptome Affected by Oncogenic RAS Signaling

The transcriptomes of human lung and kidney cells transformed with mutant KRAS to define the landscape of RAS-regulated noncoding RNAs were analyzed. It was determined that oncogenic RAS upregulates noncoding transcripts throughout the genome, many of which arise from transposable elements. These repetitive sequences are preferential targets of KRAB zinc-finger proteins, which are broadly downregulated in mutant KRAS cells and lung adenocarcinomas. Moreover, KRAS-mediated reprogramming of repetitive noncoding RNA induces an interferon response that contributes to cellular transformation. The results reveal the extent to which mutant KRAS remodels the noncoding transcriptome, expanding the scope of genomic elements regulated by this fundamental signaling pathway.

### III. Methods

Provided are methods for detecting a RAS pathway mutation in a subject. The methods include the steps of obtaining a biological sample from the subject, isolating nucleic acids from the biological sample, and analyzing the expression level of noncoding RNAs in the nucleic acids in conjunction with a corresponding reference level in a control sample, wherein a differential expression level of the noncoding RNAs compared to the corresponding reference level in the control sample indicates that the subject has a RAS pathway mutation. The method includes determining whether the subject has a KRAS mutation based on the differential expression levels of the noncoding RNAs in the biological sample of the subject compared to the expression levels of the corresponding reference genes in a control sample, optionally, from a control subject. Optionally, the RAS pathway mutation is in KRAS, NRAS, HRAS, EGFR, NFI, MET or BRAF. Optionally, the RAS pathway mutation is in KRAS. Optionally, the subject has or is suspected of having cancer. Optionally, the cancer is a RAS mutant cancer. Optionally, the RAS mutant cancer is a lung cancer, pancreatic cancer, colorectal cancer, or melanoma.

Optionally, the method further comprises analyzing the expression level of a gene involved in the interferon (IFN) alpha, IFN gamma, or KRAB-Zn Finger response. An increase in the expression level of the gene involved in the IFN alpha, IFN-gamma, or a decrease in the expression level of the gene involved in the KRAB-Zn finger response relative to a corresponding reference level for the gene in the control sample from the control subject indicates that the subject has cancer. Optionally, the method comprises analyzing the expression level of noncoding RNAs transcribed from transposable elements. Optionally, the transposable element is selected from the group consisting of a LINE-1 element, LINE-2 element, ERVK element, ERV1 element, EVRL element, EVRL-MaLR element, Alu element, hAT-Charlie element, MIR element, and combinations thereof. Optionally, the LINE-1 element is L1MC4a. Optionally, the Alu element is selected from the group consisting of AluSu, AluSg, AluJo, AluY, AluSz6, and combinations thereof. Optionally, the hAT-Charlie element is MER20. Optionally, the transposable element is a transposable element from Table 1.

TABLE 1
A list of Transposable Elements.

| | chromo- | | | | extra- | intra- | | | | |
|---|---|---|---|---|---|---|---|---|---|---|
| TE name | some | start | stop | strand | cellular | cellular | log2fc | pval | family | clade |
| AluSx3 | chr10 | 43498100 | 43498227 | − | 405.487817 | 0 | 8.6670683 | 2.37E−06 | Alu | SINE |
| MLT1D | chr9 | 12930470 | 12930648 | + | 286.038538 | 0 | 8.16510064 | 3.49E−06 | ERVL-MaLR | LTR |
| AluSz6 | chr19 | 50992223 | 50992364 | − | 249.763882 | 0 | 7.97018576 | 2.51E−06 | Alu | SINE |
| L1M1 | chrX | 57546443 | 57548317 | + | 207.873453 | 0 | 7.70648533 | 2.53E−06 | L1 | LINE |
| AluSx3 | chr2 | 201977489 | 201977642 | − | 123.339799 | 0 | 6.95814434 | 1.78E−06 | Alu | SINE |
| AluSp | chr21 | 22397624 | 22397949 | + | 89.760081 | 0 | 6.50398599 | 3.21E−06 | Alu | SINE |
| AluSz6 | chr1 | 210340205 | 210340270 | + | 74.151946 | 0 | 6.23173856 | 6.63E−07 | Alu | SINE |
| AluYj4 | chrX | 101224183 | 101224266 | − | 64.774013 | 0 | 6.03944579 | 3.27E−04 | Alu | SINE |
| L1MEc | chr11 | 43143473 | 43143667 | + | 149.432043 | 1.35167933 | 5.99927674 | 7.52E−06 | L1 | LINE |
| AluSq | chr2 | 168979572 | 168979702 | + | 62.8043653 | 0 | 5.99558323 | 3.78E−06 | Alu | SINE |
| FRAM | chrX | 71504594 | 71504800 | + | 52.407455 | 0 | 5.73896923 | 1.42E−06 | Alu | SINE |
| AluSz | chr12 | 25362124 | 25362331 | + | 52.375471 | 0 | 5.73810499 | 1.12E−06 | Alu | SINE |
| AluSc | chr4 | 38547706 | 38547786 | − | 48.3150637 | 0 | 5.62395649 | 1.59E−05 | Alu | SINE |
| L1HS | chr9 | 3398852 | 3398947 | + | 45.467272 | 0 | 5.53814304 | 1.59E−06 | L1 | LINE |
| AluYc | chr2 | 15600719 | 15600847 | − | 44.8880363 | 0 | 5.52004617 | 5.65E−07 | Alu | SINE |
| AluY | chr1 | 225667962 | 225668038 | + | 43.0497167 | 0 | 5.46106083 | 4.06E−05 | Alu | SINE |
| AluYa5 | chr5 | 88747315 | 88747648 | − | 60.4074603 | 0.463525 | 5.39089464 | 1.29E−05 | Alu | SINE |
| AluSg4 | chr21 | 8246147 | 8246456 | + | 38.7195243 | 0 | 5.31177644 | 2.81E−06 | Alu | SINE |
| AluSx3 | chr13 | 28412728 | 28412832 | − | 34.836283 | 0 | 5.1633491 | 3.61E−05 | Alu | SINE |
| AluSx | chr12 | 71248738 | 71249065 | − | 30.384463 | 0 | 4.97197862 | 1.74E−06 | Alu | SINE |
| Alusq 2 | chr10 | 80483627 | 80483961 | + | 30.1735257 | 0 | 4.96224943 | 1.89E−06 | Alu | SINE |
| AluJo | chr17 | NA | NA | + | 271.443348 | 7.940794 | 4.9294095 | 7.80E−06 | Alu | SINE |
| (GAATG)n | chr2 | 90379546 | 90379676 | + | 28.7841597 | 0 | 4.89647335 | 9.19E−07 | Satellite | Satellite |
| AluSc8 | chr16 | 4810582 | 4810760 | + | 28.3741833 | 0 | 4.87647684 | 1.44E−06 | Alu | SINE |
| AluY | chr21 | 8244370 | 8244652 | − | 28.3024913 | 0 | 4.87295142 | 4.51E−06 | Alu | SINE |
| L2a | chr19 | 50098116 | 50098329 | − | 27.5886517 | 0 | 4.83737067 | 6.14E−07 | L2 | LINE |
| L1M5 | chrX | 103377820 | 103378035 | − | 116.074286 | 3.097779 | 4.83643825 | 2.99E−05 | L1 | LINE |
| AluYe5 | chr15 | 52297045 | 52297355 | + | 27.0531323 | 0 | 4.81008996 | 4.22E−06 | Alu | SINE |
| AluSx1 | chr22 | 37661893 | 37662115 | − | 48.363461 | 0.78463767 | 4.78974045 | 7.45E−06 | Alu | SINE |
| MER11B | chr3 | 15738159 | 15739435 | − | 26.5730077 | 0 | 4.78518474 | 3.21E−06 | ERVK | LTR |
| AluSq2 | chr5 | 155975591 | 155975929 | − | 26.5351363 | 0 | 4.78320185 | 1.67E−06 | Alu | SINE |
| AluSp | chr2 | 205178024 | 205178227 | + | 26.0363917 | 0 | 4.75683071 | 5.35E−06 | Alu | SINE |
| AluJr | chr10 | 103127773 | 103127989 | − | 25.8680647 | 0 | 4.7478205 | 2.20E−06 | Alu | SINE |
| AluY | chr9 | 62501247 | 62501362 | − | 25.2815013 | 0 | 4.71597579 | 5.06E−05 | Alu | SINE |
| AluSc | chr9 | 39851510 | 39851748 | − | 24.5382757 | 0 | 4.67458921 | 2.06E−06 | Alu | SINE |
| AluSc8 | chr14 | 33328419 | 33328720 | − | 24.496676 | 0 | 4.67223727 | 1.43E−06 | Alu | SINE |
| MSTB2 | chr2 | 88521950 | 88522042 | − | 24.41919 | 0 | 4.66784615 | 1.42E−04 | ERVL-MaLR | LTR |
| AluSx1 | chr5 | 69908517 | 69908814 | − | 23.9566713 | 0 | 4.64135362 | 2.26E−06 | Alu | SINE |
| AluJr | chr7 | 74309917 | 74310238 | + | 23.6838627 | 0 | 4.62549627 | 1.93E−06 | Alu | SINE |
| AluSx3 | chr5 | 116767334 | 116767502 | + | 23.5750143 | 0 | 4.61912035 | 7.68E−07 | Alu | SINE |
| AluYe5 | chr1 | 109055068 | 109055418 | − | 22.8120043 | 0 | 4.57361716 | 5.77E−06 | Alu | SINE |
| AluSz | chr22 | NA | NA | − | 22.285492 | 0 | 4.54135946 | 7.36E−05 | Alu | SINE |
| L2b | chr7 | 7566192 | 7566577 | + | 21.8128827 | 0 | 4.51177685 | 3.34E−06 | L2 | LINE |
| AluSp | chr3 | 71900597 | 71900937 | − | 21.7123977 | 0 | 4.50540811 | 3.00E−06 | Alu | SINE |
| FAM | chr1 | 27666137 | 27666312 | + | 73.2758763 | 2.41133733 | 4.44448439 | 1.62E−05 | Alu | SINE |
| AluYc | chr21 | 23776244 | 23776394 | − | 20.7138573 | 0 | 4.44054413 | 1.18E−06 | Alu | SINE |
| AluSq2 | chr12 | 68810030 | 68810334 | + | 20.2603593 | 0 | 4.41009408 | 5.68E−06 | Alu | SINE |
| (CATTC)n | chr5 | 49666174 | 49667431 | + | 20.035275 | 0 | 4.39473877 | 3.34E−06 | Satellite | Satellite |
| AluSc8 | chr22 | 17651701 | 17651988 | − | 19.548805 | 0 | 4.36098259 | 2.86E−06 | Alu | SINE |
| Tigger17a | chr5 | 69648115 | 69648263 | − | 19.4563287 | 0 | 4.35447534 | 9.47E−07 | TcMar- | DNA |
| | | | | | | | | | Tigger | |
| AluY | chr20 | 19322925 | 19323235 | + | 19.3724127 | 0 | 4.34854494 | 4.77E−06 | Alu | SINE |
| LOR1b | chr10 | 32975216 | 32975701 | + | 19.0917463 | 0 | 4.32853106 | 1.04E−06 | ERV1 | LTR |
| AluSz6 | chr6 | 111271656 | 111271970 | + | 42.933248 | 1.22707333 | 4.30209219 | 2.94E−05 | Alu | SINE |
| AluSx3 | chr10 | 112842033 | 112842161 | − | 18.4945563 | 0 | 4.28499942 | 5.78E−06 | Alu | SINE |
| FRAM | chr9 | 81614914 | 81615136 | + | 35.553071 | 0.88625 | 4.27639981 | 7.91E−06 | Alu | SINE |
| Alu | chr13 | 89848192 | 89848272 | − | 439.825403 | 21.8256883 | 4.27147709 | 3.06E−05 | Alu | SINE |
| AluYc | chr20 | 25423442 | 25423596 | − | 18.192018 | 0 | 4.26243451 | 1.01E−06 | Alu | SINE |
| AluSq2 | chr17 | 8694586 | 8694915 | − | 18.1659867 | 0 | 4.26047637 | 1.49E−06 | Alu | SINE |
| AluYb8 | chr5 | 118118247 | 118118538 | + | 18.1384893 | 0 | 4.25840505 | 5.72E−06 | Alu | SINE |
| AluY | chr5 | 69656368 | 69656676 | + | 17.7906873 | 0 | 4.23194593 | 1.09E−04 | Alu | SINE |
| AluSc8 | chrY | NA | NA | − | 17.5773467 | 0 | 4.21547256 | 9.76E−07 | Alu | SINE |
| L1M5 | chr14 | 99611728 | 99611979 | − | 17.5240633 | 0 | 4.21132869 | 5.05E−07 | L1 | SINE |
| AluSp | chr17 | 43258769 | 43259071 | − | 32.9254997 | 0.83391533 | 4.20937112 | 1.47E−04 | Alu | SINE |
| AluYh3a3 | chr2 | 182076182 | 182076367 | − | 17.4520577 | 0 | 4.2057098 | 1.64E−06 | Alu | SINE |
| AluSz6 | chr2 | 17812776 | 17813071 | + | 17.4504183 | 0 | 4.20558162 | 6.85E−05 | Alu | SINE |
| AluYb8 | chr6 | 133050354 | 133050692 | − | 17.2358173 | 0 | 4.18870296 | 4.40E−06 | Alu | SINE |
| AluY | chr1 | 101255977 | 101256280 | + | 17.2207587 | 0 | 4.18751113 | 1.41E−06 | Alu | SINE |
| AluYf1 | chr15 | 66020711 | 66021046 | + | 17.10628 | 0 | 4.17841826 | 2.40E−06 | Alu | SINE |
| AluYa5 | chr18 | 63875755 | 63876063 | + | 17.0841637 | 0 | 4.17665497 | 3.54E−06 | Alu | SINE |
| AluSc8 | chr7 | 138165535 | 138165725 | − | 16.9110527 | 0 | 4.16277822 | 1.28E−06 | Alu | SINE |
| MIRb | chr8 | 25061881 | 25062044 | − | 16.7061827 | 0 | 4.14618131 | 1.04E−06 | MIR | SINE |
| AluSx1 | chr19 | 50092121 | 50092393 | + | 16.6644217 | 0 | 4.14277461 | 7.39E−05 | Alu | SINE |
| AluSp | chr9 | 130287463 | 130287580 | + | 16.4932663 | 0 | 4.12872779 | 5.89E−05 | Alu | SINE |
| MLT1K | chr16 | 50398411 | 50398708 | − | 16.3972803 | 0 | 4.12078989 | 8.88E−07 | ERVL-MaLR | LTR |
| L2 | chr9 | 65149930 | 65150126 | − | 16.1621393 | 0 | 4.1011575 | 1.24E−06 | L2 | LINE |
| L1MA4A | chr13 | 56621992 | 56622161 | + | 16.1535893 | 0 | 4.10043858 | 7.37E−05 | L1 | LINE |
| L1MC5 | chr1 | 109244600 | 109244886 | + | 15.9027447 | 0 | 4.07918563 | 8.48E−07 | L1 | LINE |
| AluY | chr1 | 85241559 | 85241868 | − | 15.878423 | 0 | 4.07710821 | 2.05E−06 | Alu | SINE |
| MIRb | chr1 | 27388676 | 27388828 | + | 15.7143203 | 0 | 4.06301279 | 5.65E−06 | MIR | SINE |
| AluSp | chr8 | 64665544 | 64665874 | − | 15.600004 | 0 | 4.05311168 | 1.61E−06 | Alu | SINE |
| AluYb8 | chr5 | 143974084 | 143974240 | + | 132.240695 | 7.028711 | 4.05272259 | 8.88E−05 | Alu | SINE |
| AluY | chr7 | 104974768 | 104975099 | + | 15.5632447 | 0 | 4.04991341 | 1.57E−05 | Alu | SINE |
| AluJo | chr18 | 21841985 | 21842121 | + | 15.5516517 | 0 | 4.04890328 | 9.32E−07 | Alu | SINE |
| AluSp | chrX | 138123200 | 138123380 | − | 15.4488887 | 0 | 4.03991821 | 5.24E−05 | Alu | SINE |
| AluYb8 | chr2 | 8005331 | 8005665 | − | 15.2821723 | 0 | 4.02522129 | 5.66E−06 | Alu | SINE |
| AluYg6 | chr20 | 3394938 | 3395270 | − | 14.9476763 | 0 | 3.99527433 | 1.77E−06 | Alu | SINE |
| AluY | chr9 | 129180246 | 129180548 | + | 14.9469177 | 0 | 3.99520569 | 3.80E−06 | Alu | SINE |
| AluSc | chr16 | 28803860 | 28804161 | + | 14.863981 | 0 | 3.98768295 | 4.53E−04 | Alu | SINE |
| MLT1B | chrX | 122774171 | 122774587 | + | 20.2365133 | 0.34035367 | 3.98586129 | 8.77E−06 | ERVL-MaLR | LTR |
| LTR27C | chr1 | 156301546 | 156302024 | + | 14.8387103 | 0 | 3.98538296 | 1.25E−06 | ERV1 | LTR |
| AluSc8 | chr10 | 132665461 | 132665693 | + | 14.7071547 | 0 | 3.97334996 | 6.79E−07 | Alu | SINE |
| AluSz6 | chr1 | 120080551 | 120080857 | − | 14.588976 | 0 | 3.96245426 | 9.54E−07 | Alu | SINE |
| AluY | chr18 | 65732635 | 65732890 | + | 14.5673937 | 0 | 3.96045552 | 6.98E−07 | Alu | SINE |
| Charlie3 | chr5 | 154709626 | 154710216 | − | 14.288645 | 0 | 3.93438864 | 1.70E−06 | hAT-Charlie | DNA |
| Tigger16a | chr1 | 175153259 | 175153543 | − | 14.0466873 | 0 | 3.91137399 | 7.96E−06 | TcMar- | DNA |
| | | | | | | | | | Tigger | |
| AluSx | chr7 | 17099908 | 17100215 | + | 14.0099937 | 0 | 3.90785146 | 2.04E−06 | Alu | SINE |
| AluJb | chr16 | NA | NA | − | 20.2551237 | 0.41841633 | 3.90545769 | 1.10E−05 | Alu | SINE |
| MER4A1 | chr21 | 8194987 | 8195329 | + | 13.8204727 | 0 | 3.88951955 | 1.12E−06 | ERV1 | LTR |
| L2b | chr19 | 43620173 | 43620581 | − | 13.813456 | 0 | 3.88883636 | 1.03E−06 | L2 | LINE |
| L1MB7 | chr3 | 12588153 | 12588324 | + | 13.639165 | 0 | 3.87176136 | 5.24E−07 | L1 | LINE |
| AluSx4 | chr1 | 31056600 | 31056793 | − | 13.5487923 | 0 | 3.8628275 | 2.03E−05 | Alu | SINE |
| AluSg | chr19 | 50104462 | 50104760 | + | 13.41749 | 0 | 3.84974812 | 1.36E−06 | Alu | SINE |
| AluY | chr16 | NA | NA | − | 13.3647107 | 0 | 3.84445703 | 1.92E−06 | Alu | SINE |
| L1MC4 | chr1 | 146064962 | 146065400 | + | 16.2769343 | 0.20670333 | 3.8397043 | 5.87E−06 | L1 | LINE |
| AluY | chr21 | 30304472 | 30304772 | − | 13.2820323 | 0 | 3.83612938 | 8.49E−06 | Alu | SINE |
| AluSz6 | chr22 | 43007212 | 43007515 | − | 13.2643853 | 0 | 3.83434568 | 2.83E−05 | Alu | SINE |
| AluJo | chr15 | 23312247 | 23312559 | − | 13.2090047 | 0 | 3.82873359 | 9.18E−07 | Alu | SINE |
| AluSq2 | chr1 | 92373614 | 92373928 | + | 13.121167 | 0 | 3.81978742 | 9.09E−07 | Alu | SINE |
| AluYa5 | chr11 | 124135519 | 124135829 | − | 13.057796 | 0 | 3.81329852 | 2.92E−06 | Alu | SINE |
| MIRb | chr10 | 93462370 | 93462571 | − | 12.995314 | 0 | 3.80687195 | 5.73E−05 | MIR | SINE |
| AluJb | chr2 | 203384279 | 203384609 | − | 12.9699973 | 0 | 3.80425984 | 8.25E−07 | Alu | SINE |
| L2c | chr8 | 123183995 | 123184309 | + | 12.8957633 | 0 | 3.79657318 | 3.62E−06 | L2 | LINE |
| L1M4c | chr11 | 65499662 | 65499925 | + | 410.259423 | 28.6330817 | 3.79476817 | 3.86E−04 | L1 | LINE |
| AluYk3 | chr14 | 84131085 | 84131422 | + | 16.1855297 | 0.24374167 | 3.78843555 | 5.45E−06 | Alu | SINE |
| MIRb | chr2 | 20661262 | 20661495 | − | 12.7947417 | 0 | 3.78604653 | 1.14E−06 | MIR | SINE |
| MER1B | chr4 | 52644405 | 52644717 | + | 12.7473477 | 0 | 3.7810814 | 3.26E−05 | hAT-Charlie | DNA |
| L2 | chr4 | 38658278 | 38658579 | + | 12.6796347 | 0 | 3.7739578 | 1.33E−06 | L2 | LINE |
| AluSq | chr2 | 152001407 | 152001738 | + | 12.6604203 | 0 | 3.77192997 | 8.88E−07 | Alu | SINE |
| AluJo | chr16 | 28774050 | 28774374 | − | 12.6498293 | 0 | 3.77081101 | 8.14E−07 | Alu | SINE |
| MIR | chr8 | 96187289 | 96187509 | − | 12.625055 | 0 | 3.76819015 | 6.78E−06 | MIR | SINE |
| MIR | chr5 | 67647655 | 67647877 | − | 12.6125223 | 0 | 3.76686251 | 1.97E−05 | MIR | SINE |
| LTR16 | chr1 | 81920367 | 81920785 | + | 12.5308877 | 0 | 3.75818458 | 1.38E−06 | ERVL | LTR |
| MamRep38 | chrX | 110806546 | 110806828 | − | 12.527604 | 0 | 3.75783443 | 1.33E−06 | hAT | DNA |
| L1ME4b | chr8 | 85648060 | 85648285 | − | 12.4505263 | 0 | 3.74959072 | 5.92E−07 | L1 | LINE |
| AluJo | chr1 | 31384806 | 31385118 | + | 12.442757 | 0 | 3.74875715 | 6.77E−07 | Alu | SINE |
| AluSp | chr21 | 8245775 | 8246063 | + | 21.2914233 | 0.65949067 | 3.74767632 | 1.37E−05 | Alu | SINE |
| AluJb | chr11 | 17348301 | 17348639 | − | 31.4469443 | 1.42001233 | 3.74499631 | 5.44E−05 | Alu | SINE |
| L2c | chr2 | 111286949 | 111287111 | − | 12.3876477 | 0 | 3.74283058 | 1.91E−06 | L2 | LINE |
| AluYa5 | chr1 | 122125427 | 122125746 | − | 12.343476 | 0 | 3.73806264 | 3.55E−06 | Alu | SINE |
| AluYj4 | chrX | 118908698 | 118909019 | − | 12.341187 | 0 | 3.73781513 | 1.42E−06 | Alu | SINE |
| AluSz | chr7 | 5886636 | 5886939 | − | 12.3295027 | 0 | 3.73655105 | 6.29E−06 | Alu | SINE |
| MLT2A1 | chr2 | 167943031 | 167943454 | − | 12.2413953 | 0 | 3.72698325 | 3.85E−05 | ERVL | LTR |
| AluJb | chr12 | 110463488 | 110463797 | + | 12.240648 | 0 | 3.72690182 | 5.21E−07 | Alu | SINE |
| AluJb | chr9 | 2491711 | 2492016 | + | 12.2065723 | 0 | 3.72318417 | 1.41E−06 | Alu | SINE |
| AluSz6 | chr12 | 108636777 | 108637096 | − | 12.200254 | 0 | 3.72249379 | 8.05E−07 | Alu | SINE |
| AluSz6 | chr2 | 30091087 | 30091421 | + | 12.1869447 | 0 | 3.72103843 | 2.83E−06 | Alu | SINE |
| AluYa5 | chr1 | 12838207 | 12838475 | + | 12.1791963 | 0 | 3.72019049 | 9.17E−06 | Alu | SINE |
| Tigger19b | chr11 | 75499426 | 75499599 | − | 12.0707993 | 0 | 3.70827547 | 5.95E−07 | TcMar- | DNA |
| | | | | | | | | | Tigger | |
| L1MC5 | chr19 | 17306540 | 17306854 | − | 24.7852523 | 0.972941 | 3.70812645 | 3.05E−05 | L1 | LINE |
| AluY | chr2 | 13620252 | 13620457 | − | 12.0444827 | 0 | 3.70536782 | 5.24E−07 | Alu | SINE |
| AluY | chr1 | 159738705 | 159739038 | − | 12.0165003 | 0 | 3.70226971 | 1.20E−06 | Alu | SINE |
| AluSp | chr1 | 203423709 | 203424008 | + | 11.9727753 | 0 | 3.69741525 | 5.12E−06 | Alu | SINE |
| AluSx1 | chr16 | 21413445 | 21413750 | + | 11.9089413 | 0 | 3.69029878 | 7.88E−07 | Alu | SINE |
| AluYb8 | chr11 | 17178714 | 17179024 | − | 11.8970513 | 0 | 3.68896935 | 1.55E−05 | Alu | SINE |
| AluYc3 | chr12 | 131350548 | 131350870 | − | 11.7903963 | 0 | 3.67698906 | 1.40E−05 | Alu | SINE |
| L2b | chr16 | 16352344 | 16352546 | + | 11.7359807 | 0 | 3.67083815 | 1.65E−05 | L2 | LINE |
| AluSx1 | chr11 | 73191981 | 73192288 | − | 11.7352663 | 0 | 3.67075723 | 8.25E−07 | Alu | SINE |
| AluSx1 | chr1 | 21628271 | 21628437 | + | 11.698224 | 0 | 3.66655483 | 6.43E−05 | Alu | SINE |
| AluSx1 | chr17 | 65607928 | 65608248 | + | 11.6792897 | 0 | 3.66440202 | 2.17E−06 | Alu | SINE |
| AluYa5 | chr2 | 66634421 | 66634752 | + | 19.197313 | 0.60448833 | 3.65397817 | 2.90E−05 | Alu | SINE |
| L2c | chr1 | 214568453 | 214568792 | + | 11.572037 | 0 | 3.65214652 | 9.05E−07 | L2 | LINE |
| AluSg | chr19 | 47928122 | 47928435 | − | 11.5018597 | 0 | 3.64407081 | 0.00139313 | Alu | SINE |
| L2 | chr19 | 29921583 | 29921655 | − | 50.5093113 | 3.13473867 | 3.6389652 | 1.80E−05 | L2 | LINE |
| AluY | chr7 | 81481617 | 81481924 | − | 11.436043 | 0 | 3.63645561 | 9.55E−07 | Alu | SINE |
| AluSz6 | chr7 | 74800883 | 74801192 | + | 11.4102343 | 0 | 3.63345845 | 2.15E−06 | Alu | SINE |
| AluYb9 | chr2 | 157576831 | 157577154 | − | 11.405015 | 0 | 3.63285157 | 4.01E−05 | Alu | SINE |
| AluJo | chrX | 28483285 | 28483439 | + | 11.399692 | 0 | 3.63223238 | 7.98E−05 | Alu | SINE |
| AluY | chr5 | 34195560 | 34195856 | + | 11.3908483 | 0 | 3.63120306 | 3.29E−06 | Alu | SINE |
| AluSc | chr8 | 28960748 | 28961084 | − | 11.3716637 | 0 | 3.62896761 | 1.50E−06 | Alu | SINE |
| AluSx | chr7 | 73083723 | 73084052 | − | 11.3680717 | 0 | 3.62854868 | 3.00E−05 | Alu | SINE |
| AluSc | chr11 | 56883098 | 56883420 | − | 11.3555197 | 0 | 3.62708379 | 8.91E−07 | Alu | SINE |
| L2a | chr16 | 54232756 | 54233062 | − | 11.3271987 | 0 | 3.62377308 | 3.17E−06 | L2 | LINE |
| AluYg6 | chr17 | 58836740 | 58837039 | + | 11.2540303 | 0 | 3.61518442 | 3.48E−05 | Alu | SINE |
| AluSx | chr17 | 77132227 | 77132522 | − | 11.1985437 | 0 | 3.60863702 | 4.03E−06 | Alu | SINE |
| AluY | chr7 | 39812382 | 39812732 | + | 15.4661977 | 0.35886533 | 3.59903306 | 6.05E−06 | Alu | SINE |
| AluY | chr1 | 111211467 | 111211776 | − | 11.067333 | 0 | 3.59303496 | 1.07E−04 | Alu | SINE |
| L1HS | chr12 | 83450360 | 83450836 | + | 11.0271033 | 0 | 3.58821731 | 2.58E−06 | L1 | LINE |
| AluSx1 | chr12 | 100806509 | 100806835 | − | 10.9861247 | 0 | 3.58329338 | 7.20E−06 | Alu | SINE |
| AluSx1 | chr19 | 47955897 | 47956171 | − | 10.9331333 | 0 | 3.576901 | 9.84E−07 | Alu | SINE |
| AluSx3 | chr7 | 6624748 | 6625052 | − | 10.9323753 | 0 | 3.57680936 | 1.96E−05 | Alu | SINE |
| AluYb9 | chr16 | 5897017 | 5897334 | − | 10.9223367 | 0 | 3.57559511 | 4.19E−06 | Alu | SINE |
| AluSx1 | chr3 | 11506467 | 11506763 | + | 10.919644 | 0 | 3.57526924 | 7.15E−07 | Alu | SINE |
| AluSp | chr17 | 7749027 | 7749364 | + | 10.9178947 | 0 | 3.5750575 | 1.06E−06 | Alu | SINE |
| AluSq | chr5 | 176895077 | 176895376 | − | 10.9005487 | 0 | 3.57295618 | 2.39E−06 | Alu | SINE |
| AluSx1 | chr16 | 28351371 | 28351673 | + | 10.8665603 | 0 | 3.56882991 | 2.62E−04 | Alu | SINE |
| MLT1J1 | chr8 | 7601844 | 7602187 | − | 10.859566 | 0 | 3.56797931 | 4.00E−05 | ERVL-MaLR | LTR |
| AluJb | chr2 | 46578451 | 46578763 | + | 10.8215627 | 0 | 3.56334885 | 5.81E−07 | Alu | SINE |
| LTR16B1 | chr1 | 223703455 | 223703792 | + | 10.7924477 | 0 | 3.55979129 | 1.05E−04 | ERVL | LTR |
| L1PA16 | chr1 | 210374768 | 210375002 | − | 10.7596593 | 0 | 3.55577436 | 6.22E−07 | L1 | LINE |
| AluSx | chr22 | 45637465 | 45637788 | − | 10.7413557 | 0 | 3.55352709 | 8.60E−07 | Alu | SINE |
| MER41A | chr12 | 118308025 | 118308407 | + | 10.7162997 | 0 | 3.55044509 | 1.48E−06 | ERV1 | LTR |
| AluSq2 | chr17 | 7812640 | 7812964 | + | 10.653685 | 0 | 3.54271432 | 1.14E−05 | Alu | SINE |
| AluSz6 | chr7 | 73001533 | 73001839 | − | 10.649637 | 0 | 3.5422131 | 6.68E−05 | Alu | SINE |
| AluJo | chr20 | 4122024 | 4122215 | + | 10.6455357 | 0 | 3.5417051 | 6.97E−07 | Alu | SINE |
| L2a | chr3 | 33144674 | 33144879 | − | 23.8162083 | 1.13341933 | 3.54004324 | 7.17E−06 | L2 | LINE |
| AluSx3 | chr8 | 127221617 | 127221975 | + | 10.6032607 | 0 | 3.53645837 | 1.13E−06 | Alu | SINE |
| L1MD2 | chr5 | 137234007 | 137234565 | − | 10.5872123 | 0 | 3.53446162 | 6.41E−04 | L1 | LINE |
| MIRc | chr15 | 84436027 | 84436258 | − | 10.545227 | 0 | 3.52922463 | 1.96E−05 | MIR | SINE |
| MIRb | chr10 | 103850888 | 103851059 | − | 10.5258783 | 0 | 3.52680479 | 1.14E−05 | MIR | SINE |
| AluSx1 | chr7 | 5510024 | 5510332 | + | 10.5130257 | 0 | 3.52519512 | 8.01E−07 | Alu | SINE |
| L2a | chr2 | 208026158 | 208026361 | + | 10.440014 | 0 | 3.51601691 | 3.42E−06 | L2 | LINE |
| AluSz | chr5 | 68033480 | 68033789 | + | 10.4176263 | 0 | 3.51319085 | 5.86E−07 | Alu | SINE |
| AluJb | chr19 | 50097260 | 50097455 | + | 10.3996757 | 0 | 3.51092087 | 4.08E−05 | Alu | SINE |
| AluJr | chr17 | 57903680 | 57903951 | + | 10.3985183 | 0 | 3.5107744 | 3.48E−05 | Alu | SINE |
| AluSq2 | chr20 | 50626740 | 50627034 | + | 10.3862827 | 0 | 3.50922492 | 3.49E−06 | Alu | SINE |
| Alusg | chr10 | 88049874 | 88050204 | + | 18.4782793 | 0.713225 | 3.5070797 | 3.37E−05 | Alu | SINE |
| MIR | chrX | 47143797 | 47144003 | + | 10.3672653 | 0 | 3.50681332 | 9.64E−06 | MIR | SINE |
| L1PA11 | chr3 | 16335414 | 16335795 | + | 10.3576617 | 0 | 3.50559394 | 9.46E−07 | L1 | LINE |
| AluSx1 | chr1 | 154391983 | 154392293 | + | 10.3405567 | 0 | 3.50341955 | 1.40E−06 | Alu | SINE |
| AluSx1 | chr20 | 50430102 | 50430412 | − | 10.3381483 | 0 | 3.50311314 | 1.61E−06 | Alu | SINE |
| AluSc8 | chr16 | 29524575 | 29524882 | − | 10.3376557 | 0 | 3.50305045 | 8.82E−06 | Alu | SINE |
| AluSx | chr16 | 28451076 | 28451374 | − | 10.327902 | 0 | 3.50180878 | 1.73E−06 | Alu | SINE |
| MIRb | chr5 | 139474115 | 139474347 | − | 10.3120383 | 0 | 3.49978701 | 8.57E−06 | MIR | SINE |
| AluJb | chr7 | 124441848 | 124442196 | − | 14.1908007 | 0.34711433 | 3.49525371 | 6.69E−06 | Alu | SINE |
| AluY | chr12 | 46681584 | 46681921 | + | 10.2626787 | 0 | 3.49347809 | 2.77E−06 | Alu | SINE |
| AluSp | chr13 | 19795207 | 19795539 | + | 15.080628 | 0.429881 | 3.49135676 | 1.31E−05 | Alu | SINE |
| AluYk3 | chr5 | 36695339 | 36695643 | − | 10.2291573 | 0 | 3.48917776 | 2.86E−06 | Alu | SINE |
| AluSx1 | chr1 | 52569111 | 52569411 | + | 10.1720667 | 0 | 3.48182418 | 2.74E−06 | Alu | SINE |
| AluSz | chr14 | 55209123 | 55209462 | − | 17.0886543 | 0.62221933 | 3.47904429 | 1.25E−05 | Alu | SINE |
| LTR16B2 | chr2 | 39107197 | 39107626 | + | 10.1278537 | 0 | 3.47610345 | 5.46E−07 | ERVL | LTR |
| AluSg7 | chr11 | 64319999 | 64320306 | + | 13.7317927 | 0.324079 | 3.47587189 | 4.11E−05 | Alu | SINE |
| AluSx1 | chr7 | 74766207 | 74766506 | + | 10.1143573 | 0 | 3.47435262 | 1.69E−05 | Alu | SINE |
| L1PA13 | chr1 | 240267296 | 240267648 | + | 10.0937257 | 0 | 3.47167205 | 4.15E−05 | L1 | LINE |
| AluJo | chr3 | 124896467 | 124896763 | + | 10.0775433 | 0 | 3.46956607 | 1.31E−04 | Alu | SINE |
| AluSp | chr9 | 97272790 | 97273138 | − | 10.0512953 | 0 | 3.46614357 | 1.67E−06 | Alu | SINE |
| AluSx1 | chr7 | 5183609 | 5183928 | − | 10.0455727 | 0 | 3.46539631 | 1.24E−04 | Alu | SINE |
| AluSc | chr1 | 24419808 | 24420102 | + | 10.0435833 | 0 | 3.46513646 | 7.45E−07 | Alu | SINE |
| L2 | chr20 | 47362448 | 47362715 | + | 10.0280597 | 0 | 3.46310707 | 5.07E−05 | L2 | LINE |
| Tigger3b | chr3 | 122351736 | 122351988 | + | 10.0164633 | 0 | 3.46158924 | 4.80E−05 | TcMar- | DNA |
| | | | | | | | | | Tigger | |
| AluY | chr13 | NA | NA | − | 12.9097187 | 0.28186367 | 3.4397785 | 1.06E−05 | Alu | SINE |
| AluYb8 | chr6 | 21191987 | 21192320 | − | 16.6775323 | 0.67581233 | 3.3989844 | 3.04E−04 | Alu | SINE |
| L2b | chr11 | 73875322 | 73875639 | − | 20.563587 | 1.07345767 | 3.37848669 | 4.66E−05 | L2 | LINE |
| L2c | chr1 | 173417953 | 173418208 | + | 14.9607233 | 0.56860667 | 3.34697049 | 1.39E−05 | L2 | LINE |
| Tigger1 | chr9 | 41234464 | 41234772 | − | 23.2187877 | 1.393879 | 3.33870451 | 4.23E−05 | TcMar- | DNA |
| | | | | | | | | | Tigger | |
| AluYa8 | chr3 | 75065217 | 75065561 | − | 13.2826833 | 0.42640333 | 3.32381316 | 3.85E−05 | Alu | SINE |
| L2a | chr1 | 154586460 | 154586894 | − | 10.5702707 | 0.15825967 | 3.32039199 | 1.13E−05 | L2 | LINE |
| AluSx | chr1 | 200378820 | 200379149 | − | 16.105337 | 0.75586733 | 3.28419078 | 3.75E−05 | Alu | SINE |
| AluY | chr5 | 176411895 | 176412196 | + | 11.7438067 | 0.31717567 | 3.27427662 | 4.20E−05 | Alu | SINE |
| AluY | chr21 | 41115488 | 41115736 | − | 16.2063087 | 0.79702633 | 3.25925417 | 9.41E−06 | Alu | SINE |
| AluSz | chr17 | 6873941 | 6874278 | + | 13.2957603 | 0.508295 | 3.24459682 | 1.98E−05 | Alu | SINE |
| AluYh3a3 | chr21 | 37213531 | 37213810 | + | 11.8538423 | 0.37037567 | 3.22955634 | 5.75E−06 | Alu | SINE |
| AluYg6 | chr6 | 38954423 | 38954732 | + | 10.281821 | 0.20841233 | 3.22281523 | 9.63E−06 | Alu | SINE |
| LTR5 | chr11 | 41305799 | 41306022 | + | 20.3373943 | 1.31644933 | 3.20339698 | 1.33E−05 | ERVK | LTR |
| HERVIP10FH- | chr21 | 8229744 | 8229975 | − | 13.493196 | 0.67671867 | 3.11166322 | 2.58E−05 | ERV1 | LTR |
| int | | | | | | | | | | |
| AluSx | chr16 | 61353234 | 61353434 | + | 102.155945 | 11.0720277 | 3.09508705 | 2.77E−04 | Alu | SINE |
| AluJr | chr6 | 79816762 | 79817059 | + | 14.047042 | 0.76607767 | 3.09085921 | 2.27E−05 | Alu | SINE |

Optionally, analyzing the expression level of the noncoding RNAs comprises performing polymerase chain reaction (PCR), reverse transcriptase polymerase chain reaction (RT-PCR), single-cell RNA-sequencing, microarray analysis, a Northern blot, serial analysis of gene expression (SAGE), immunoassay, hybridization capture, cDNA sequencing, direct RNA sequencing, nanopore sequencing, mass spectrometry, a CRISPR based technology, or combinations thereof. Optionally, measuring the expression level of the noncoding RNAs comprises performing sequencing. Optionally, the sequencing comprises obtaining one or more sequencing reads of the noncoding RNAs. Optionally, the analyzing comprises aligning the sequencing reads of the noncoding RNAs to repetitive sequences in a human genome.

The biological sample can include extracellular vesicles isolated from cells from the subject. Optionally, the nucleic acids from the biological sample includes polyadenylated RNAs. Optionally, the biological sample is a blood sample, a urine sample, a saliva sample, or a tissue sample. Optionally, the biological sample is a tissue sample. The subject can be a mammal, e.g., a human.

Optionally, the methods further include administering to the subject one or more anticancer agents. Optionally, the anticaner agent is an inhibitor of a RAS pathway gene. Optionally, the anticancer agent is an inhibitor of KRAS.

In the provided methods, if the gene in the biological sample from the subject displays a differential expression level relative to the corresponding reference gene in the control sample from the control subject, i.e., higher or lower than the expression level of the gene in the control sample by at least 2%, 4%, 6%, 8%, 10%, 20%, 30%, 40%, or 50%, then the subject may have cancer and/or a KRAS mutation. In certain embodiments, the cancer and/or the KRAS mutation may be in a tissue of the subject (e.g., lung).

In some embodiments, the method comprises analyzing the expression level of one or more genes involved in the interferon (IFN) alpha or gamma response. The expression level of one or more genes involved in the IFN alpha or gamma response can increase in response to a KRAS mutation. In yet other embodiments, the method comprises analyzing the expression level of a gene encoding a KRAB zinc-finger (KZNF) protein. The expression level of a gene encoding a KZNF protein can decrease in response to a KRAS mutation.

In the methods described herein, in some embodiments, the subject is suspected of having a RAS pathway mutation, e.g., a RAS pathway mutation is in a lung, colorectal, or pancreas tissue of the subject.

In the methods described herein, in some embodiments, the cancer is a lung cancer (e.g., lung adenocarcinoma). The cancer may be characterized by an oncogenic defect in the RAS pathway. In particular embodiments, the oncogenic defect comprises an activating mutation in KRAS.

### IV. Inhibitors

In some embodiments of the methods described herein, an increased expression level of a noncoding RNA in a biological sample from a subject compared to a corresponding reference expression level of the same gene in a control sample from a control subject may indicate that the subject has cancer. In some embodiments of the methods described herein, once it is determined that a subject (e.g., a subject suspected of having cancer) has an increased expression level of the gene relative to a control sample, the subject may be administered a therapeutically effective amount of an inhibitor to inhibit the expression level of the gene.

An inhibitor of the gene refers to an agent that inhibits or decreases the expression level and/or the activity of the gene. An inhibitor may inhibits or decreases the transcription of the gene, binds to the gene, and/or inhibits interaction between the gene and another protein or nucleic acid. In some embodiments, an inhibitor may be an inhibitory RNA (e.g., small interfering RNA (siRNA), an antisense RNA, microRNA (miRNA), and short hairpin RNA (shRNA)), an aptamer, an antibody, a CRISPR RNA or a small molecule.

In some embodiments, an inhibitor may be an inhibitory RNA, e.g., small interfering RNA (siRNA), an antisense RNA, microRNA (miRNA), a CRISPR RNA or short hairpin RNA (shRNA). In some embodiments, the inhibitory RNA targets a sequence that is identical or substantially identical (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical) to a target sequence in the gene. A target sequence in the gene may be a portion of the gene comprising at least 20, at least 30, at least 40, at least 50, at least 60, at least 70, at least 80, at least 90, or at least 100 contiguous nucleotides, e.g., from 20-500, 20-250, 20-100, 50-500, or 50-250 contiguous nucleotides.

In some embodiments of the methods described herein, once it is determined that a subject (e.g., a subject suspected of having cancer) has an increased expression level of one or more noncoding RNAs relative to a control sample, the subject may be administered a therapeutically effective amount of an siRNA that inhibits or decreases the expression level of the gene. An siRNA may be produced from a short hairpin RNA (shRNA). A shRNA is an artificial RNA molecule with a hairpin turn that can be used to silence target gene expression via the siRNA it produces in cells. See, e.g., Fire et. al., Nature 391:806-811, 1998; Elbashir et al., Nature 411:494-498, 2001; Chakraborty et al., Mol Ther Nucleic Acids 8:132-143, 2017; and Bouard et al., Br. J. Pharmacol. 157:153-165, 2009. Expression of shRNA in cells is typically accomplished by delivery of plasmids or through viral or bacterial vectors. Suitable bacterial vectors include but not limited to adeno-associated viruses (AAVs), adenoviruses, and lentiviruses. After the vector has integrated into the host genome, the shRNA is then transcribed in the nucleus by polymerase II or polymerase III (depending on the promoter used). The resulting pre-shRNA is exported from the nucleus, then processed by Dicer and loaded into the RNA-induced silencing complex (RISC). The sense strand is degraded by RISC and the antisense strand directs RISC to an mRNA that has a complementary sequence. A protein called Ago2 in the RISC then cleaves the mRNA, or in some cases, represses translation of the mRNA, leading to its destruction and an eventual reduction in the protein encoded by the mRNA. Thus, the shRNA leads to targeted gene silencing.

In some embodiments, once it is determined that a subject (e.g., a subject suspected of having cancer) has an increased expression level of one or more noncoding RNAs relative to a control sample, the subject may be administered a therapeutically effective amount of an shRNA capable of hybridizing to a portion of the gene. The shRNA may be encoded in a vector. In some embodiments, the vector further comprises appropriate expression control elements known in the art, including, e.g., promoters (e.g., inducible promoters or tissue specific promoters), enhancers, and transcription terminators.

In some embodiments, once it is determined that a subject (e.g., a subject suspected of having cancer) has an increased expression level of one or more genes in Tables 1-3 relative to a control sample, the subject may be administered a therapeutically effective amount of an siRNA capable of hybridizing to a portion of the gene. The siRNA may be encoded in a vector. In some embodiments, the vector further comprises appropriate expression control elements known in the art, including, e.g., promoters (e.g., inducible promoters or tissue specific promoters), enhancers, and transcription terminators.

### V. Detecting Expression Levels

Techniques and methods for measuring the expression levels of genes are available in the art. For example, detection and/or quantification of noncoding RNAs may be accomplished by any one of a number methods or assays employing recombinant DNA or RNA technologies known in the art, including but not limited to, polymerase chain reaction (PCR), single-cell RNA-sequencing, reverse transcription PCR (RT-PCR), microarrays, Northern blot, serial analysis of gene expression (SAGE), immunoassay, hybridization capture, cDNA sequencing, direct RNA sequencing, nanopore sequencing, CRISPR based technology, and mass spectrometry.

In some embodiments, hybridization capture methods may be used for detection and/or quantification of the noncoding RNAs. Some examples of hybridization capture methods include, e.g., capture hybridization analysis of RNA targets (CHART), chromatin isolation by RNA purification (ChIRP), CRISPR based technology and RNA affinity purification (RAP). In general, cells and tissues expressing the RNA of interest can be cross-linked and solubilized by shearing. The RNA of interest can then be enriched using rationally designed biotin tagged antisense oligonucleotides. The captured RNA complexes can then be rinsed and eluted. The eluted material can be analyzed for the molecules of interest. The associated RNAs are commonly analyzed with qPCR or high throughput sequencing, and the recovered proteins can be analyzed with Western blots or mass spectrometry. General techniques for performing hybridization capture methods are described in the art and can be found in, e.g., Machyna and Simon, Briefings in Functional Genomics 17(2):96-103, 2018, which is incorporated herein by reference in its entirety. Further, Li et al, JCI Insight. 3(7):e98942, 2018 also describes methods of studying RNA (e.g., extracellular RNA) and is incorporated herein by reference in its entirety.

In some embodiments, microarrays may be used to measure the expression levels of the genes. An advantage of microarray analysis is that the expression of each of the genes can be measured simultaneously, and microarrays can be specifically designed to provide a diagnostic expression profile for a particular disease or condition (e.g., cancer). Microarrays may be prepared by selecting probes which comprise a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic nucleic acids. The polynucleotide sequences of the probes may also be synthesized nucleotide sequences, such as synthetic oligonucleotide sequences. Probes may be immobilized to a solid support which may be either porous or non-porous. For example, the probes may be polynucleotide sequences which are attached to a nitrocellulose or nylon membrane or filter covalently at either the 3′ or the 5′ end of the polynucleotide. Such hybridization probes are well-known in the art (see, e.g., Sambrook, et al., Molecular Cloning: A Laboratory Manual (3rd Ed., 2001). In one embodiment, a microarray may include a support or surface with an ordered array of binding (e.g., hybridization) sites or “probes” each representing one of the genes described herein. More specifically, each probe of the array may be located at a known, predetermined position on the solid support such that the identity (i.e., the sequence) of each probe can be determined from its position in the array (i.e., on the support or surface). Each probe may be covalently attached to the solid support at a single site.

Quantitative reverse transcriptase PCR (qRT-PCR) can also be used to determine the expression profiles of the genes. The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMY-RT) and Moloney murine leukemia virus reverse transcriptase (MLVRT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, CA, USA), following the manufacturer's instructions. The derived cDNA can then be used as a template in the subsequent PCR reaction. Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TAQMAN PCR typically utilizes the 5′-nuclease activity of Taq polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, may be designed to detect nucleotide sequence located between the two PCR primers. The probe is non-extendible by Taq DNA polymerase enzyme, and may be labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

Serial Analysis Gene Expression (SAGE) can also be used to determine RNA expression level. SAGE analysis does not require a special device for detection, and may be used for simultaneously detecting the expression of a large number of transcription products. First, RNA is extracted, converted into cDNA using a biotinylated oligo (dT) primer, and treated with a four-base recognizing restriction enzyme (Anchoring Enzyme: AE) resulting in AE-treated fragments containing a biotin group at their 3′ terminus. Next, the AE-treated fragments are incubated with streptavidin for binding. The bound cDNA is divided into two fractions, and each fraction is then linked to a different double-stranded oligonucleotide adapter (linker) A or B. These linkers are composed of: (1) a protruding single strand portion having a sequence complementary to the sequence of the protruding portion formed by the action of the anchoring enzyme, (2) a 5′ nucleotide recognizing sequence of the IIS-type restriction enzyme (cleaves at a predetermined location no more than 20 bp away from the recognition site) serving as a tagging enzyme (TE), and (3) an additional sequence of sufficient length for constructing a PCR-specific primer. The linker-linked cDNA is cleaved using the tagging enzyme, and only the linker-linked cDNA sequence portion remains, which is present in the form of a short-strand sequence tag. Next, pools of short-strand sequence tags from the two different types of linkers are linked to each other, followed by PCR amplification using primers specific to linkers A and B. As a result, the amplification product is obtained as a mixture comprising myriad sequences of two adjacent sequence tags (ditags) bound to linkers A and B. The amplification product is treated with the anchoring enzyme, and the free ditag portions are linked into strands in a standard linkage reaction. The amplification product is then cloned. Determination of the clone's nucleotide sequence can be used to obtain a readout of consecutive ditags of constant length. The presence of the gene corresponding to each tag can then be identified from the nucleotide sequence of the clone and information on the sequence tags.

### VI. Anticancer Agents

In methods described herein, a subject may be administered one or more anticancer agents alone or in combination with one or more inhibitors that inhibit the expression levels of one or more noncoding RNAs. An anticancer agent may be a RAS pathway inhibitor, a cytotoxic agent, a chemotherapeutic agent, or an immunosuppressive agent. An anticancer agent may be a natural or synthetic agent. In some embodiments, an anticancer agent may be capable of treating cancer, activating immune response, and/or reducing tumor load. In some embodiments, an anticancer agent may inhibit the proliferation of and/or kill cancer cells. An anticancer agent may be a small molecule, a peptide, or a protein. In some embodiments, an anticancer agent may be an agent that inhibits and/or down regulates the activity of a protein that prevents immune cell activation or a protein that exerts immunosuppressive effects.

Examples of anticancer agents include, but are not limited to, RAS pathway inhibitors such as the mutant KRAS specific inhibitors including Sotorasib/AMG 510 (LUMARKRAS™), Adagrasib (MRTX849), MRTX1133, and GDC-6036; alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and C B1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Nicolaou et al. Angew. Chem Intl. Ed. Engl., 33: 183-186 (1994)); CDP323, an oral alpha-4 integrin inhibitor; dynemicin, including dynemicin A; an esperamicin; neocarzinostatin chromophore and related chromoprotein enediyne antibiotic chromophores), aclacinomysins, actinomycin, authramycin, azaserine, bleomycin, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); combretastatin; folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, 5-azacytidine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′, 2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®, Bristol-Myers Squibb Oncology, Princeton, N.J.), albumin-engineered nanoparticle formulation of paclitaxel (ABRAXANE™), and docetaxel (TAXOTERE®, Rhome-Poulene Rorer, Antony, France); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R) (e.g., erlotinib (Tarceva™)); and VEGF-A that reduce cell proliferation; vaccines such as THERATOPE® vaccine and gene therapy vaccines, for example, ALLOVECTINO vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g. celecoxib or etoricoxib), proteosome inhibitor (e.g. PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors; tyrosine kinase inhibitors; serine-threonine kinase inhibitors such as rapamycin (sirolimus, RAPAMUNE®); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

In some embodiments, an anticancer agent is cisplatin, carboplatin, oxaliplatin, bleomycin, mitomycin C, calicheamicins, maytansinoids, doxorubicin, idarubicin, daunorubicin, epirubicin, busulfan, carmustine, lomustine, semustine, methotrexate, 6-mercaptopurine, fludarabine, 5-azacytidine, pentostatin, cytarabine, gemcitabine, 5-fluorouracil, hydroxyurea, etoposide, teniposide, topotecan, irinotecan, chlorambucil, cyclophosphamide, ifosfamide, melphalan, bortezomib, vincristine, vinblastine, vinorelbine, paclitaxel, or docetaxel.

### Chemotherapeutic Agent

In some embodiments, the anticancer agent is a chemotherapeutic agent. In some embodiments, chemotherapeutic agents may kill cancer cells or inhibit cancer cell growth. Chemotherapeutic agents may function in a non-specific manner, for example, inhibiting the process of cell division known as mitosis. Examples of chemotherapeutic agents include, but are not limited to, antimicrotubule agents (e.g., taxanes and vinca alkaloids), topoisomerase inhibitors and antimetabolites (e.g., nucleoside analogs acting as such, for example, Gemcitabine), mitotic inhibitors, alkylating agents, antimetabolites, antitumor antibiotics, mitotic inhibitors, anthracyclines, intercalating agents, agents capable of interfering with a signal transduction pathway, agents that promote apoptosis, proteosome inhibitors, and alike.

Alkylating agents are most active in the resting phase of the cell. These types of drugs are cell-cycle non-specific. Exemplary alkylating agents include, but are not limited to, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Antitumor antibiotics are chemotherapeutic agents obtained from natural products produced by species of the soil fungus, e.g., Streptomyces. These drugs act during multiple phases of the cell cycle and are considered cell-cycle specific. There are several types of antitumor antibiotics, including but are not limited to anthracyclines (e.g., Doxorubicin, Daunorubicin, Epirubicin, Mitoxantrone, and Idarubicin), chromomycins (e.g., Dactinomycin and Plicamycin), mitomycin, and bleomycin.

Antimetabolites are types of chemotherapeutic agents that are cell-cycle specific. When cells incorporate these antimetabolite substances into the cellular metabolism, they are unable to divide. This class of chemotherapeutic agents include folic acid antagonists such as Methotrexate; pyrimidine antagonists such as 5-Fluorouracil, Foxuridine, Cytarabine, Capecitabine, and Gemcitabine; purine antagonists such as 6-Mercaptopurine and 6-Thioguanine; Adenosine deaminase inhibitors such as Cladribine, Fludarabine, Nelarabine and Pentostatin.

Exemplary anthracyclines that can be used include, e.g., doxorubicin (Adriamycin® and Rubex®); Bleomycin (Lenoxane®); Daunorubicin (dauorubicin hydrochloride, daunomycin, and rubidomycin hydrochloride, Cerubidine®); Daunorubicin liposomal (daunorubicin citrate liposome, DaunoXome®); Mitoxantrone (DHAD, Novantrone®); Epirubicin (Ellence); Idarubicin (Idamycin®, Idamycin PFS®); Mitomycin C (Mutamycin®); Geldanamycin; Herbimycin; Ravidomycin; and Desacetylravidomycin.

Antimicrotubule agents include vinca alkaloids and taxanes. Exemplary vinca alkaloids include, but are not limited to, vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®). Exemplary taxanes that can be used include, but are not limited to paclitaxel and docetaxel. Non-limiting examples of paclitaxel agents include nanoparticle albumin-bound paclitaxel (ABRAXANE, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel methyl 2-glucopyranosyl succinate, see Liu et al., Bioorganic & Medicinal Chemistry Letters (2007) 17:617-620).

Exemplary proteosome inhibitors that can be used include, but are not limited to, Bortezomib (Velcade®); Carfilzomib (PX-171-007, (S)-4-Methyl-N-((S)-1-(((S)-4-methyl-1-((R)-2-methyloxiran-2-yl)-1-oxope-ntan-2-yl) amino)-1-oxo-3-phenylpropan-2-yl)-2-((S)-2-(2-morpholinoacetamid-o)-4-phenylbutanamido)-pentanamide); marizomib (NPI-0052); ixazomib citrate (MLN-9708); delanzomib (CEP-18770); and O-Methyl-N-[(2-methyl-5-thiazolyl)carbonyl]-L-seryl-O-methyl-N-[(1S)-2-[(-2R)-2-methyl-2-oxiranyl]-2-oxo-1-(phenylmethyl)ethyl]-L-serinamide (ONX-0912).

In some embodiments, the chemotherapeutic agent is selected from the group consisting of chlorambucil, cyclophosphamide, ifosfamide, melphalan, streptozocin, carmustine, lomustine, bendamustine, uramustine, estramustine, carmustine, nimustine, ranimustine, mannosulfan busulfan, dacarbazine, temozolomide, thiotepa, altretamine, 5-fluorouracil (5-FU), 6-mercaptopurine (6-MP), capecitabine, cytarabine, floxuridine, fludarabine, gemcitabine, hydroxyurea, methotrexate, pemetrexed, daunorubicin, doxorubicin, epirubicin, idarubicin, SN-38, ARC, NPC, campothecin, topotecan, 9-nitrocamptothecin, 9-aminocamptothecin, rubifen, gimatecan, diflomotecan, BN80927, DX-895 If, MAG-CPT, amsacrine, etoposide, etoposide phosphate, teniposide, doxorubicin, paclitaxel, docetaxel, gemcitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, gemcitabine, Irinotecan, albumin-bound paclitaxel, Oxaliplatin, Capecitabine, Cisplatin, docetaxel, irinotecan liposome, and etoposide, and combinations thereof.

In certain embodiments, the chemotherapeutic agent is administered at a dose and a schedule that may be guided by doses and schedules approved by the U.S. Food and Drug Administration (FDA) or other regulatory body, subject to empirical optimization.

In still further embodiments, more than one chemotherapeutic agent may be administered simultaneously, or sequentially in any order during the entire or portions of the treatment period. The two agents may be administered following the same or different dosing regimens.

### EXAMPLES

### Example 1—Materials and Methods

Cell Lines. The AALE stable cell lines pBABE-mCherry Puro (control) and pBABE-FLAGKRAS(G12D) Zeo (mutant KRAS) were generated using retroviral transduction, followed by selection in puromycin of zeocin, respectively, 2 days post-infection. Both lines were cultured in SABM Basal Medium (Lonza SABM basal medium) with added supplements and growth factors (Lonza SAGM SingleQuot Kit Suppl. & Growth Factors). AALE cell lines were maintained using Lonza's Reagent Pack subculture reagents. The HA1E cell lines were generated using lentiviral transduction (pLX317) to generate control and mutant HA1E pLX317-KRAS(G12V) stable cell lines using puromycin selection, and cells were cultured in MEM-alpha (Invitrogen) with 10% FBS (Sigma) and 1% penicillin/streptomycin (Gibco). All cell lines tested negative for mycoplasma.

siRNA Knockdowns. AALEs were seeded at 1×10⁶ cells per well of a 6-well plate in complete growth medium, then reverse transfected with 30 pmol siRNA using RNAiMAX lipofectamine according to manufacturer's protocol. Cells were grown for 3 days in transfection medium under standard culture conditions and then harvested for RNA isolation and qPCR as previously described.

Cell Viability Assay. 2×10⁴ cells were subtracted from each siRNA transfection well at the time of transfection and seeded into individual wells of an ultra-low adhesion 96-well plate. The cells were grown in standard culture conditions for 4 days. They were then harvested, and ATP production was measured using the Cell TiterGLO Luminescent Cell Viability Assay (Promega) following the manufacturer's protocol. Luminescence was measured on a Perkin Elmer VICTOR light 1420 Luminescence Counter.

RNA Isolation & Purification. For AALE cell lines, bulk RNA was isolated from cells using Quick-RNA MiniPrep kit (Zymogen). All RNA was quantified via NanoDrop-8000 Spectrophotometer. For HA1E cell lines, bulk RNA was isolated using RNeasy Mini Kit (Qiagen) and quantified via Qubit RNA BR assay kit (Thermo).

qPCR. cDNA was transcribed from 1 μg RNA using iScript cDNA Synthesis Kit (Bio-Rad) according to manufacturer protocol. cDNA was diluted 1:6 and run with iTaq Universal SYBR Green Supermix (Bio-Rad) on ViiA 7 Real-Time PCR System according to manufacturer protocol. Cycle Threshold (CT) values were converted using Standard analysis. Values obtained for target genes were normalized to HPRT.

RNA-seq. For AALE cell lines, 1 μg of total RNA was used as input for the TruSeq Stranded mRNA Sample Prep Kit (Illumina) according to manufacturer protocol. Library quality was determined through the High Sensitivity DNA Kit on a Bioanalyzer 2100 (Agilent Technologies). Multiplexed libraries were sequenced as HiSeq400 100PE runs. For HA1E cell lines, 1 μg of total RNA was used for mRNA enrichment with Dynabeads mRNA DIRECT kit (Thermo). First strand cDNA was generated with AffinityScript Multiple Temperature reverse transcriptase with oligo dT primers. Second strand cDNA was generated with mRNA Second Strand Sythesis Module (New England Biolab). DNA was cleaned up with Agencourt AMPure XP beads twice. Qubit dsDNA High Sensitivity Assay was used for concentration measurement. 1 ng of dsDNA was further subjected to library preparation with Nextera XT DNA sample prep kit (Illumina) per manufacturer instructions. Library size distribution was confirmed with Bioanalyzer (Agilent). Multiplexed libraries were sequenced as NextSeq500 75PE runs.

Single-cell RNA-seq. For single cell RNAseq, 1×10⁶ cells were harvested and resuspended in 1 mL 1×PBS/0.04% BSA (1000 cells/μl) according to the cell preparation guidelines in the 10× Genomics Chromium Single Cell 3′ Reagent Kit User Guide. GEMs were generated from an input of 3,500 cells. We used the 10× Genomics Chromium Single Cell 3′ Reagent Kits version 2 for both the GEM generation and subsequent library preparation and followed the manufacturer's reagent kit protocol. Quantification of all RNAseq libraries was performed by QB3 at UC Berkeley. RNAseq libraries were sequenced as HiSeq4000 100PE runs.

ATAC-seq. 100,000 cells were collected and centrifuged at 500×g for 5 minutes at 4C. Pellets were washed with ice-cold PBS and centrifuged. Pellets were resuspended in icecold lysis buffer. Tagmentation reaction and purification were conducted according to manufacturer's protocol (Active Motif). Libraries were sequenced on a NextSeq500 as 2×75 paired end reads.

Extracellular RNA. The exoRNeasy serum/plasma maxi kit (Qiagen) was used to isolate extracellular vesicles, which were quantified using Nanoparticle Tracking Analysis (Malvern, UK). 30 ml of cell culture supernatant was filtered to remove particles larger than 0.8 μm. The filtrate was precipitated with kit buffer and filtered through a column to collect extracellular vesicles. These vesicles were then lysed with QIAzol® lysis reagent. Total RNA was isolated using the indicated phase separation method and used to make libraries for RNA-seq, which were sequenced on a NextSeq500.

Exosomal RNA. Exosomes were isolated using the Exosome Total Isolation Chip (ExoTIC) as previously described (23). The ExoTIC device was first flushed with 2 mL of 1×PBS buffer. Then, the EVs from culture media were isolated as follows: a five millilitervolume of culture medium was drawn up in the same syringe and connected with the ExoTIC device. This syringe along with the ExoTIC device, was fixed onto a syringe pump. A pump flow rate of 5 mL/h was applied to filter the culture media, concentrating EVs in front of the nanoporous membrane. Free proteins, nucleic acids, etc., which are smaller than the membrane pore size (˜50 nm) pass through the filter pores. The EVcontaining retentate was then washed by running 5 mL of 1× PBS through the device using the same syringe. The ExoTIC device was then disconnected from the syringe, and the purified EV solution was collected via the device inlet using a 200 μL pipet. RNA extracted from the purified EV sample using the miRNeasy Mini Kit (Qiagen) was used to make libraries for RNA-seq, which were sequenced on a NextSeq500.

Statistical Analysis. All quantitative data for functional assays has been reported as means±standard deviation. Statistical significance for these was calculated using a t-test and p-values <0.05 were considered significant.

Data and Code Availability. All code for figures, file parsing, and data processing is available via https:github.com/rreggiar/aale.kras. Sequencing data is accessible via GEO accession GSE120566. Additionally, UCSC Genome Browser tracks used in figures is accessible at https://gnome.ucsc.edu/s/rreggiar/kras.and.ctrl.atac.

RNA-seq Analysis. All fastq files were trimmed with Trimmomatic 2 (0.38) (27) using the Illumina NextSeq PE adapters. The resulting trimmed files were assessed with FastQC (28) and then passed through the following analytical pipeline: Salmon (1.0): pseudoalignment of RNA-seq reads performed with Salmon (29) using the following arguments: -validateMappings -gcBias -seqBias -numBootstraps 20 using an index created from the GENCODE version 32 transcriptome fasta file using standard arguments. STAR (2.7.3a): trimmed reads were aligned to the Human genome with default arguments using a 2-pass approach described previously (30). The resulting .sam files were converted to bam, sorted, and indexed using Samtools with default arguments for all procedures (31). Sleuth (0.30.0): transcript differential expression was performed using Sleuth (32) and Wasabi (1.0.1) to convert the Salmon output into the proper format. Upon completion, the transcripts with q-values below 0.05 in the likelihood-ratio test were used to filter salmon output from which log2fc was manually calculated and paired to the sleuth output. Sleuth was primarily used for quantifying DE of Transposable Element loci in which case the provided reference was the repeat masked loci sequences from the UCSC Genome Browser. DESeq2 (1.24.0): Salmon output was imported into a DESeq object using tximport (33) and differential expression analysis was performed with standard arguments (34). All results were filtered to have padj <=0.01. In the case where R could only generate 0.00 for the padj values, they were reset to the lowest non-zero padj value in the data set. Where count data was used, it was normalized across samples using DESeq.

Transposable Element Content Analysis. Exon and 5′/3′ UTR Overlap: a whole genome .gtf file was downloaded from the UCSC Genome Browser table browser utility. This file was parsed and merged with the GENCODE v.29 reference transcriptome. This modified .gtf (now a .bed file) was passed to bedtools (35) where the overlap function was used with the following arguments: -a modified.gtf bed -b all.ucsc.rmsk.genes.bed -wao -s>retained.overlap.bed alongside a whole genome .gtf retrieved as described above except generated from the repeat-masked browser track. The resulting overlapped bed file was processed and visualized using custom R scripts. Differential Expression: Differential transcript abundance was determined using the Salmon and Sleuth procedures described above provided with a custom index comprising both the GENCODE version 29 transcripts and all transcripts extracted from the Hammel lab GTF file as described in the single cell procedures. Sleuth output was filtered and visualized using R and Tidyverse.

Zinc Finger Protein Analysis. ChIP-exo data and supplementary information were extracted from supplementary data provided by Imbeault et al (24). ZNF genes were cross referenced with DESeq2 and RepeatMasker (36) outputs to extract relevant differential expression data of ZNF proteins and Transposable Element transcripts using R. RepeatMasker output from promoter analyses was cross referenced with ChIP-exo target data to identify potential regulatory targets of differentially expressed KZNFs. Only KZNF targets with ‘score’ [see Imbeault et al] >=75 were kept for analysis. Analysis of all data was performed and visualized in R using custom scripts.

Gene Set Enrichment Analysis. Genes determined to be significantly differentially expressed in DESeq2 output were first ‘pre-ranked’ in R by the following metric: Score metric=sin(log2FoldChange) *-log10(p-value) The resulting ranked files objects were processed using the R package fgsea alongside gene set files downloaded from msigdb using the R package msigdbr. Additional code was written for select vizualizations.

Gene Ontology Analysis. Upregulated gene names were extracted from DESeq2 output using bash command line tools. Name lists were pasted into the Gene Ontology Consortium's Enrichment Analysis tool powered by PANTHER. Output data was exported as .txt files and parsed using bash command line tools. Parsed data was visualized using custom R scripts.

Single Cell Analysis. Cell Ranger: Single cell output data was processed using 10× pipeline CellRanger. The mkfastq functionality was used to generate fastq files for further downstream analysis. Salmon—Alevin: fastq files generated above were passed to Salmon alevin with the default arguments for CHROMIUM V2 data. alevin was used to psuedoalign the libraries to both the GENCODE v32 combined with the repeat masked loci sequences extracted from Hg38 via the UCSC Genome Browser. A salmon index was built from this reference with standard arguments. These alevin output matrices were imported into R using tximport. STAR-solo: This feature within the STAR software was used to generate single cell SAM files for downstream processing. Run with the recommended arguments. Seurat (3.0): Normalization and UMAP clustering were performed with Seurat following their described approach optimized to our data set (see code notebook). Additional code was written to extract count data from Seurat single cell objects using the SingleCellExperiment R package (37).

TCGA ZNF Analysis. TCGA-LUAD and GTEX lung phenotype and normalized count data were downloaded from the UCSC Xena browser TOIL data repository (https://xenabrowser.net/datapages/?cohort=TCGA%20TARGET%20GTEx&addHub=https%3A%2Fxenatreehousegiuscs.edu&removeHub=https%3A%2Fxena.treehouse.gi.ucsc.edu%3A443). The files were combined and patients were grouped by their KRAS mutation status and identity. These data were compared to and visualized alongside of data generated from our analysis using custom R code. Significance was determined with a one-way t test implemented in the R t test( ) function.

RNA Editing. RNAEditinglndexer: Single cell SAM files were subsampled into 3 equal subsets per cluster based on barcode. Each SAM file was then converted into a BAM as described above and used as input for the RNAEditinglndexer script with bed files generated by extracting the locations of detected Transposable Element loci from Sleuth output (21).

ATAC-seq Analysis. ENCODE: The ENCODE ATAC-seq pipeline was used for alignment, quality control, MACS analysis with default arguments to produce output for downstream analysis. HOMER: Narrow peaks files produced above were processed using a variety of HOMER tools with default arguments where not explicitly stated: findMotifsGenome was used to find enriched motifs in each ATAC library and their subsets. annotatePeaks was used to generate detailed annotations of the motifs found above and their context. It was also used to create gene set differential accessibility histograms by using the -hist argument set to 1. mergePeaks allowed us to identify unique and overlapping peaks across the two libraries. makeTagDirectory was used to generate peak height estimates to be applied on all histogram comparisons (38).

Quantification and statistical analysis. All statistical analyses were performed with R (version 3.6.1) running from the Rocker ‘Tidyverse’ Docker container (rocker/tidyverse:3.6.1). Unpaired, bi-directional t test was performed with the t.test( ) function on samples with 3 biological or technical replicates. Linear regression was carried out with the lm( )function.

Additional Code. All analysis was performed in the R programming language with supplemental scripts written in Bash.

### Example 2—Transcriptomic Signatures of Mutant KRAS

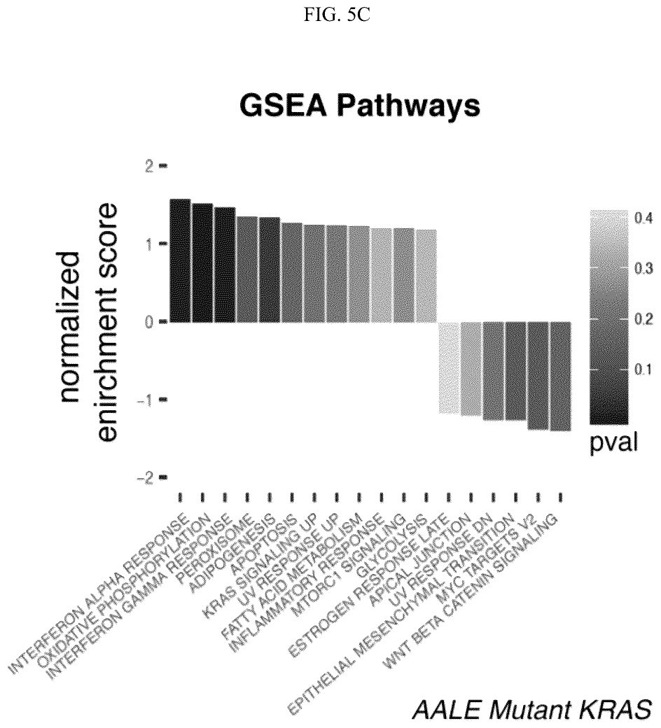

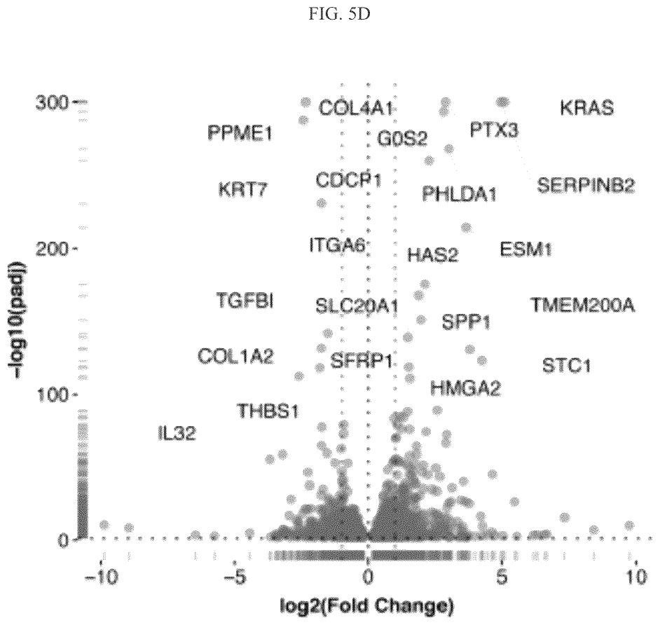

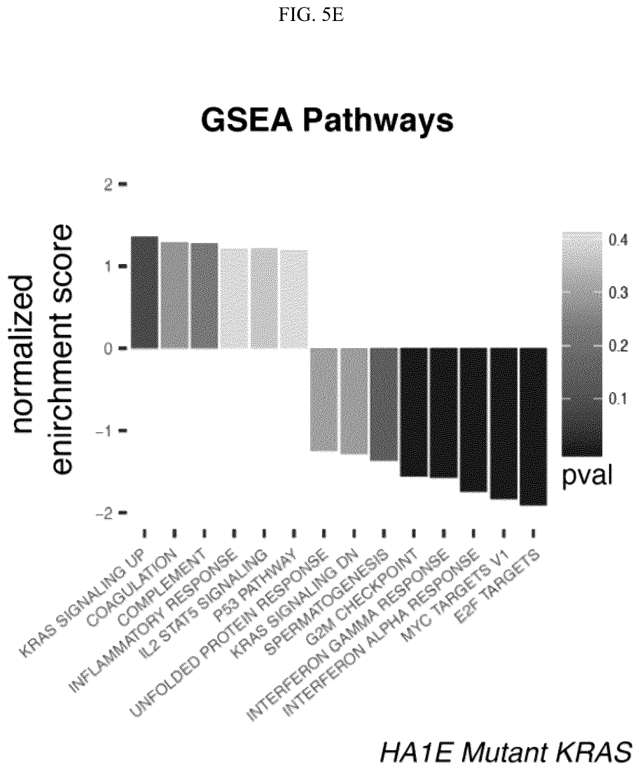

To determine the transcriptomic landscape of protein-coding and noncoding RNAs regulated by oncogenic RAS signaling, RNA sequencing (RNA-seq) was performed on human airway epithelial cells (AALE) that undergo malignant transformation upon introduction of mutant KRAS (FIG. 5A) (14). We compared the transcriptomes of AALE cells transduced with control lentiviral vector to AALEs that were transduced and transformed by mutant KRAS-containing lentiviral vector and performed differential expression analysis. We identified many protein-coding RNAs that were significantly differentially expressed (n=4323 upregulated, n=4711 down-regulated), as well as hundreds of differentially expressed lncRNAs (n=279 upregulated, n=409 downregulated), revealing the broad extent to which mutant KRAS reprograms the transcriptome (FIG. 5B). For differentially expressed genes in mutant KRAS versus control AALEs, a large proportion of upregulated lncRNAs (−75%) contained TE sequences in their exons, suggesting that repetitive noncoding loci in the genome are preferentially misregulated during mutant KRAS-mediated malignant transformation.

### Example 3—Mutant KRAS Induces an Intrinsic IFN-Stimulated Gene Signature

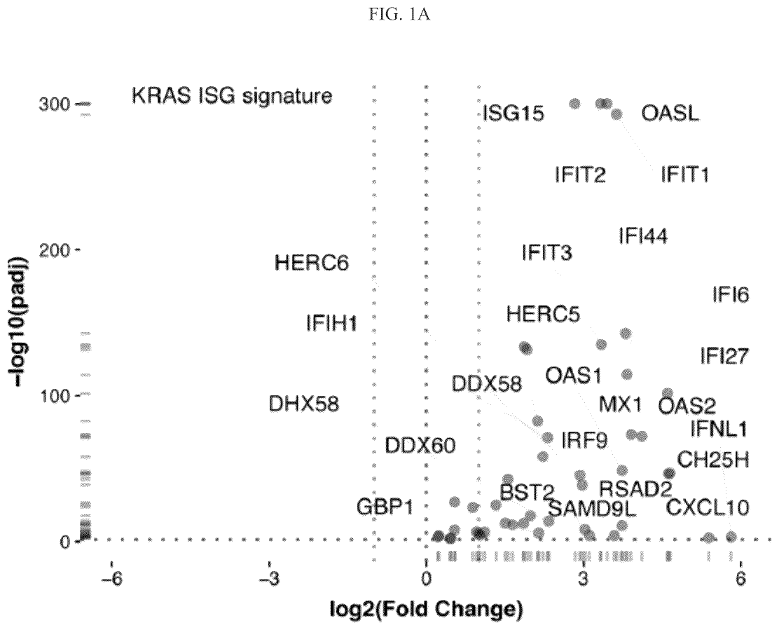

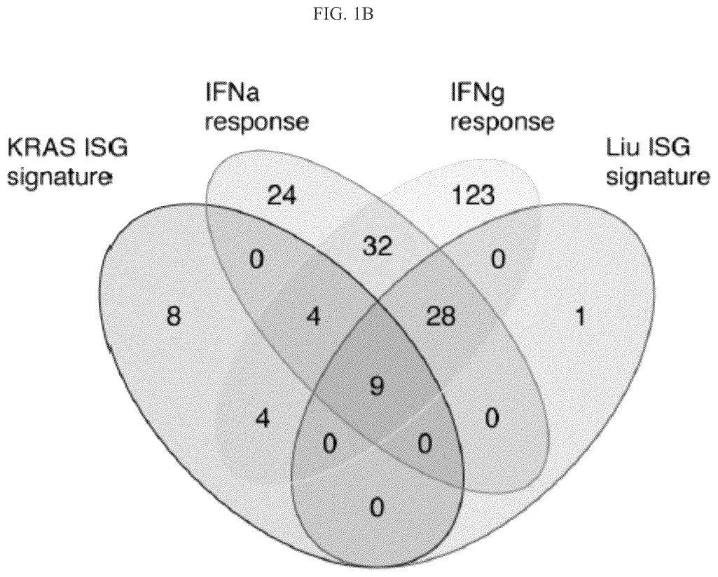

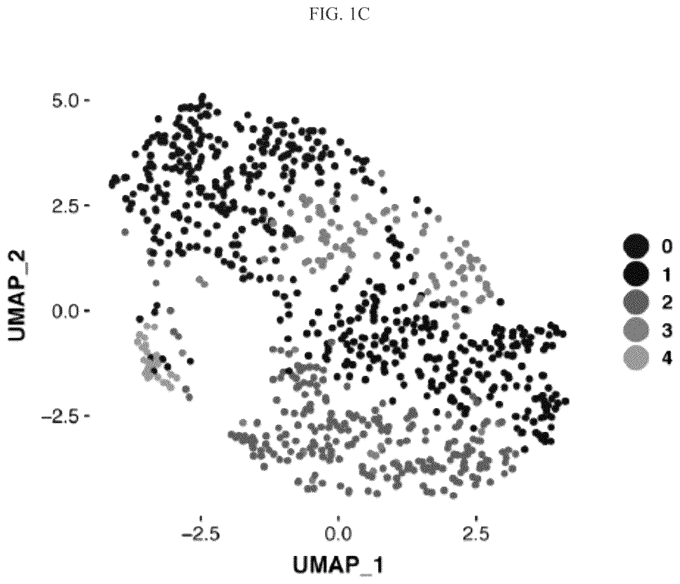

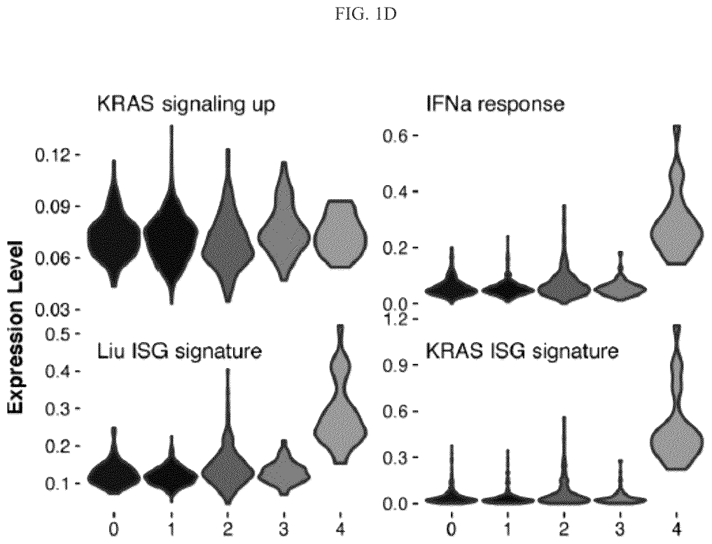

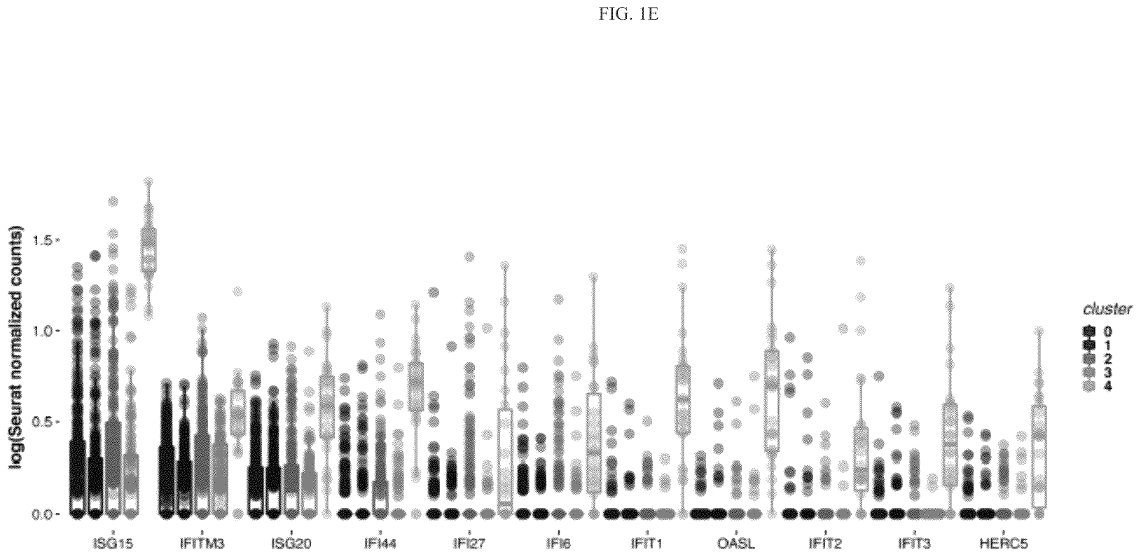

To explore the biological pathways that are perturbed by oncogenic RAS signaling, gene set enrichment analysis (GSEA) (15) was performed using genes that were differentially expressed in our mutant KRAS AALE cells. GSEA revealed that the most significantly enriched pathway was the interferon (IFN) alpha response, while the third most enriched pathway was the IFN gamma response (FIG. 5C). All known ISGs that were expressed in our mutant KRAS AALEs were examined. Most of these genes were significantly upregulated, yielding a 25-gene mutant KRAS ISG signature (FIG. 1A). The KRAS ISG signature was then compared to IFN alpha (INFa) and IFN gamma (IFNg) response genes, along with the ADAR-dependent ISG signature described in Liu et al. (16). While the majority of KRAS ISG signature genes overlapped with the other 3 IFN-related signatures, several genes were unique to mutant KRAS cells (FIG. 1B). These results reveal that mutant KRAS signaling activates an intrinsic ISG response in transformed AALEs.

It was then investigated whether this mutant KRAS ISG signature was specific to lung cells or if other cell types responded similarly. RNAseq was performed on human embryonic kidney cells (HA1E) that were primed for oncogenic KRAS driven transformation (17) and analyzed how mutant KRAS altered their transcriptomes. Similar to transformed AALEs, thousands of protein-coding RNAs (n=2635 up, n=2639 down) and hundreds of lncRNAs were upregulated (n=165) or downregulated (n=223) (FIG. 5D). GSEA, however, showed no enrichment for any IFN pathways in mutant KRAS-transformed HA1E cells, even though they were significantly enriched for upregulated KRAS signaling. In contrast to the mutant KRAS AALEs, both IFNg and IFNa response pathways were among the most significantly downregulated gene sets in mutant KRAS HA1Es (FIG. 5E), highlighting the cell type-specific differences in how the transcriptome is reprogrammed by mutant KRAS.

To assess the heterogeneity of the KRAS ISG signature in mutant KRAS AALEs, single-cell RNA-seq (scRNA-seq) (n=1503 cells) (FIG. 1C) was performed. While KRAS signaling genes were upregulated in each cluster of cells, there was extensive heterogeneity in the IFNa response, Liu ISG, and KRAS ISG signatures, with the highest levels present in cluster 4 (FIG. 1D). This was also consistent at the gene level for ISGs (FIG. 1E), and these results indicate that individual cells respond differently to oncogenic KRAS signaling.

### Example 4—Epigenetic Reprogramming of ISGs By Mutant KRAS

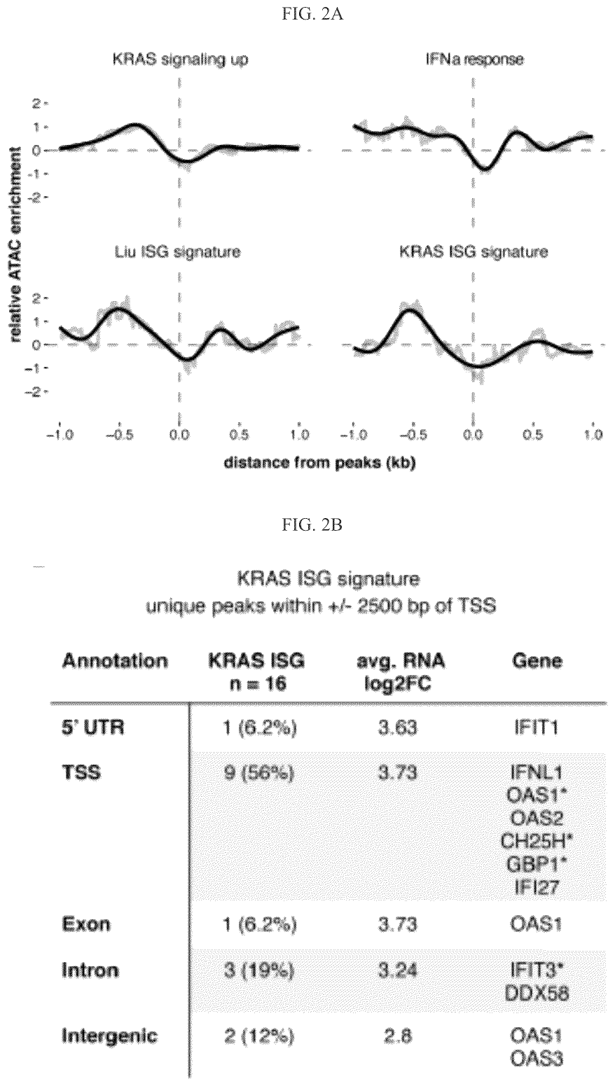

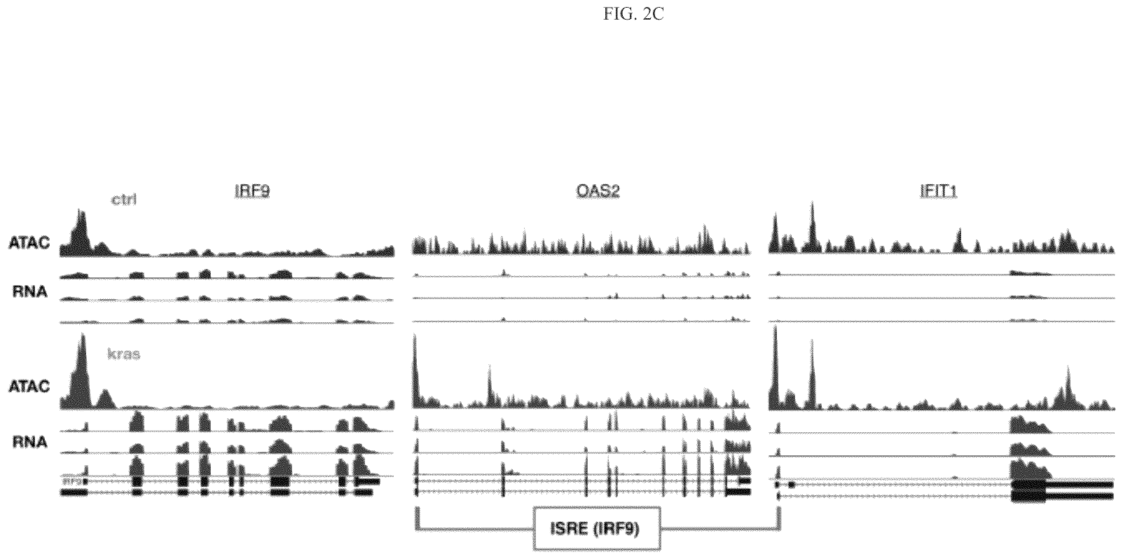

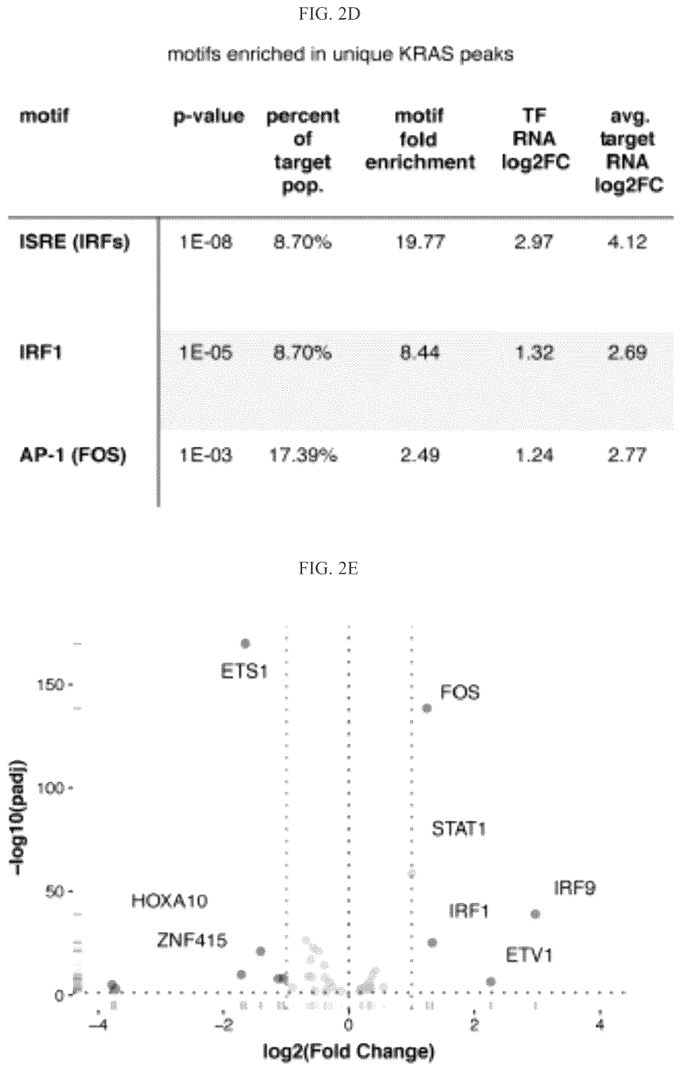

To elucidate the molecular mechanisms involved in inducing intrinsic ISG signatures in mutant KRAS AALE cells, an Assay for Transposase-Accessible Chromatin using sequencing (ATAC-seq) (18) was performed. In mutant KRAS AALEs, open chromatin was strongly enriched at gene promoters for upregulated KRAS signaling genes, as well as KRAS ISG signature genes (FIG. 2A). Open chromatin peaks were enriched at transcriptional start sites (TSS) of the ISGs OAS1, CH25H, GBP1, and IFNL1, all of which were significantly and specifically upregulated by mutant KRAS signaling (FIG. 2B). Several of these KRAS ISGs, including OAS2 and IFIT1, contained interferon-stimulated response elements (ISRE) that are recognized by the transcription factor IRF9, which also undergoes epigenetic activation in mutant KRAS AALEs (FIG. 2C). Some of the most enriched motifs at ATAC-seq peaks in mutant KRAS cells were for IRF9, IRF1, and AP-1 (FOS) (FIG. 2D), and genes at these open loci were significantly upregulated, as were the transcription factors that bind to these enriched motifs (FIG. 2E). These results reveal that oncogenic KRAS signaling induces intrinsic ISG expression through the epigenetic activation of ISG promoters.

### Example 5—Mutant KRAS Upregulates Transposable Element-Derived Noncoding RNAs

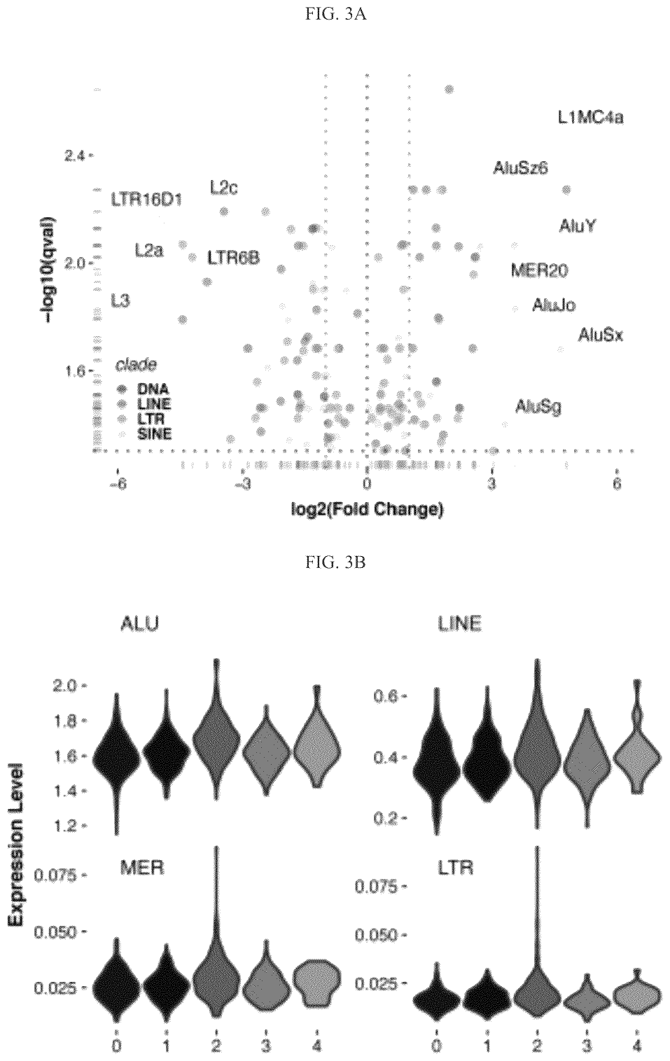

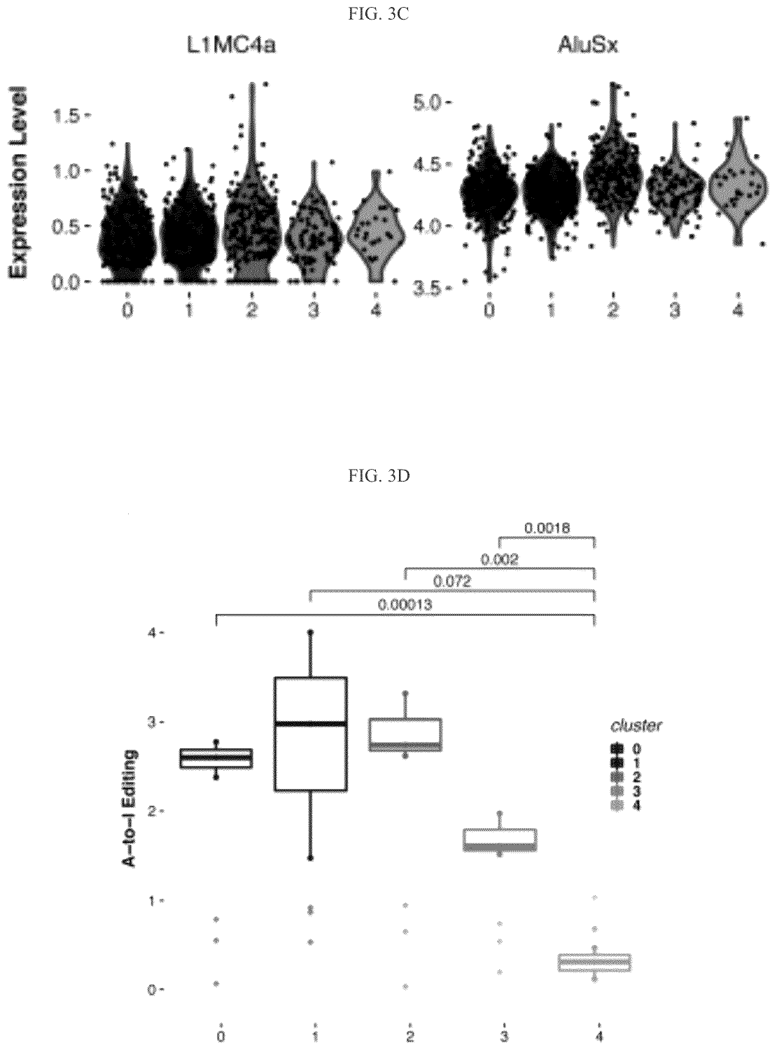

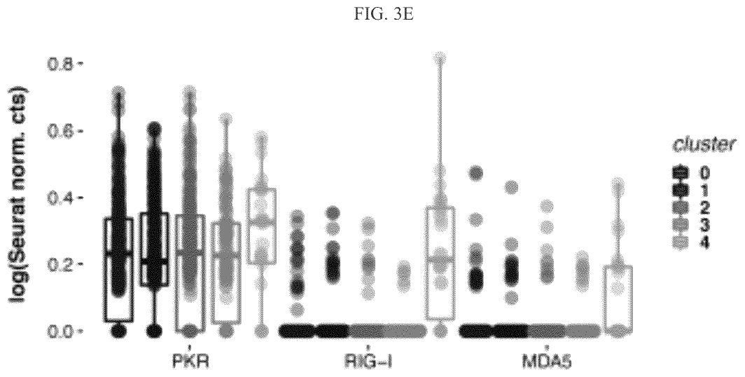

The molecular basis for intrinsic ISG signature activation in mutant KRAS AALE cells was investigated by analyzing the abundance of repetitive noncoding RNAs transcribed from TEs, which induce an IFN response in cancer cells when aberrantly expressed (19, 20). The LINE-1 element L1MC4a, the Alu elements AluSx, AluSg, AluJo, AluY, and AluSz6, and the hAT-Charlie element MER20 were all significantly upregulated in mutant KRAS AALE cells (FIG. 3A), suggesting that oncogenic KRAS signaling induces an ISG signature in transformed lung cells through the activation of TE-derived noncoding RNAs.

TE expression heterogeneity was examined in our single-cell RNA-seq data from mutant KRAS AALEs. Substantial heterogeneity was not observed in ALU, LINE, MER, or LTR class TE expression (FIG. 3B), as well as for the specific TEs L1MC4a and AluSx (FIG. 3C). RNA editing analysis on the scRNA-seq data to look for Ato-I editing (21), revealed TE-derived double-stranded RNAs (dsRNAs) exhibited significantly lower levels of RNA editing in cluster 4 (FIG. 3D), even though TE RNA levels were similar across all of the scRNA-seq data clusters. Mutant KRAS cells with lower levels of RNA editing exhibited higher expression of the dsRNA sensors MDA5 and RIG-I, while all of the clusters showed relatively similar levels of PKR expression, with a slightly higher level of PKR expression in the cells with lower RNA editing (FIG. 3E). These results reveal the extent of single cell heterogeneity in both RNA editing and dsRNA sensor ISGs in mutant KRAS cells.

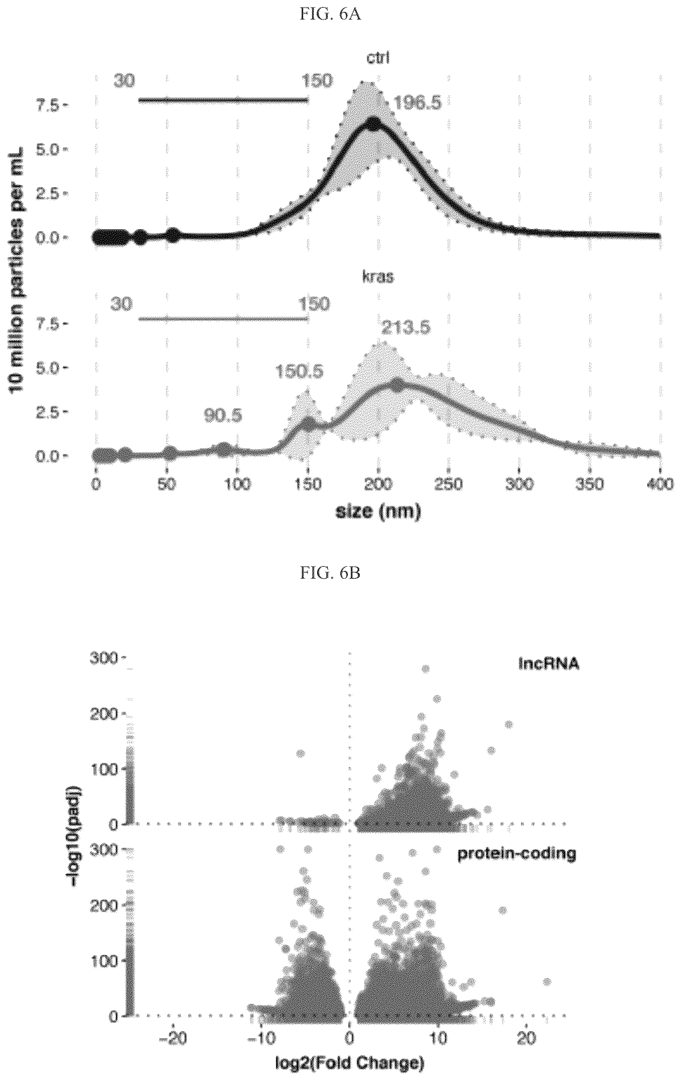

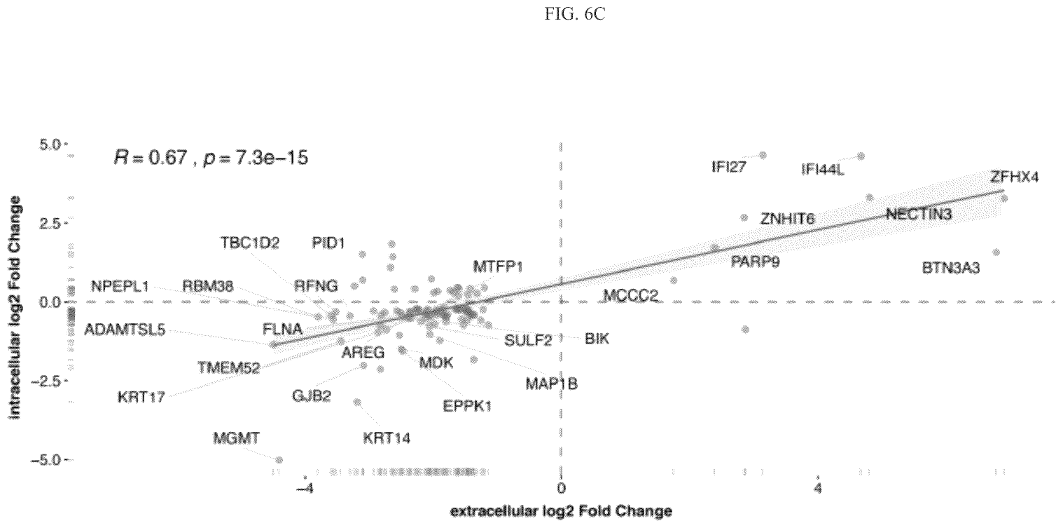

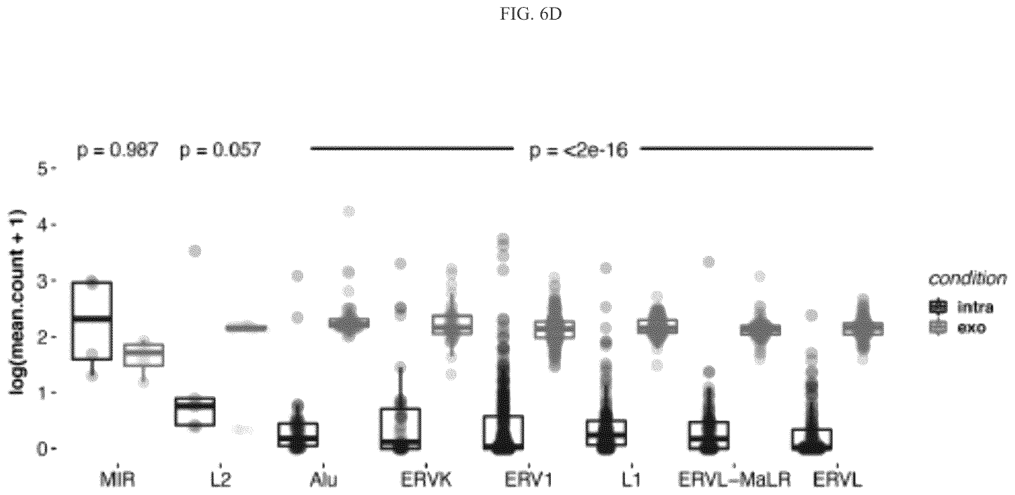

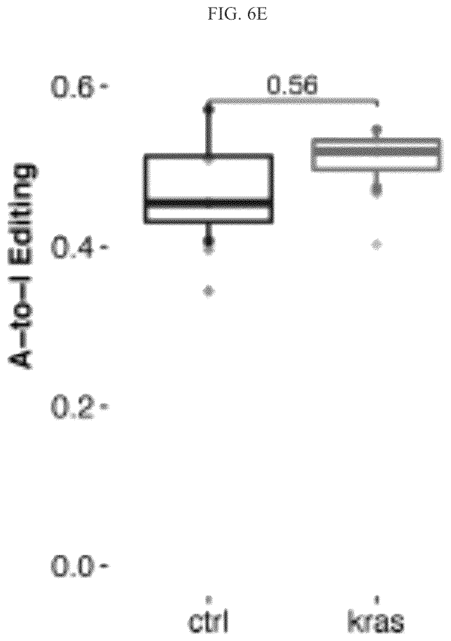

To test whether extracellular RNAs that are released from mutant KRAS cells might also exhibit differential RNA editing, extracellular vesicles were isolated from the culture media of control and mutant KRAS AALEs (22, 23). Extracellular vesicles isolated from mutant KRAS AALE cell culture media were comprised of two different sized classes of vesicles that were ˜150 nm and ˜213 nm in diameter, while vesicles from control AALE media were ˜196 nm in size (FIG. 6A). RNA-seq showed extracellular vesicles were enriched in lncRNAs when compared to the intracellular RNA composition (FIG. 6B). We also observed strong correlation between differentially expressed genes in intracellular and extracellular RNAseq data (FIG. 6C). Moreover, repetitive noncoding RNAs derived from Alu, ERV, and L1 elements were significantly enriched in extracellular vesicles (FIG. 6D) but did not exhibit differential RNA editing (FIG. 6E), suggesting that intrinsic KRAS ISG signatures in mutant KRAS AALEs are not significantly affected by TE-derived repetitive noncoding RNAs that are packaged into extracellular vesicles.

### Example 6—Broad Epigenetic Silencing of KRAB Zinc-Finger Genes by Mutant KRAS

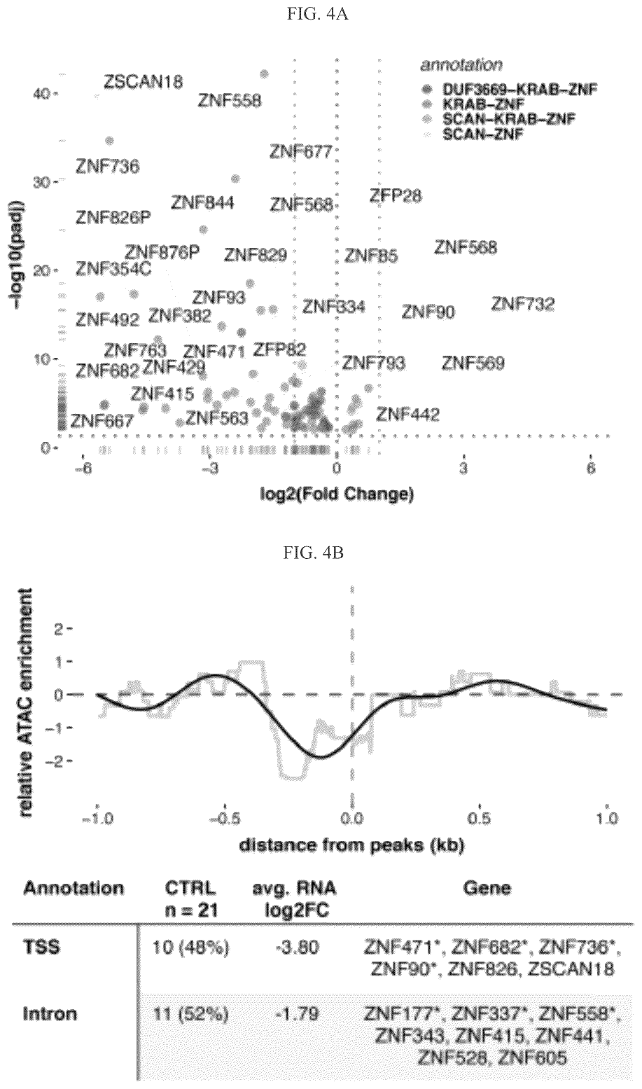

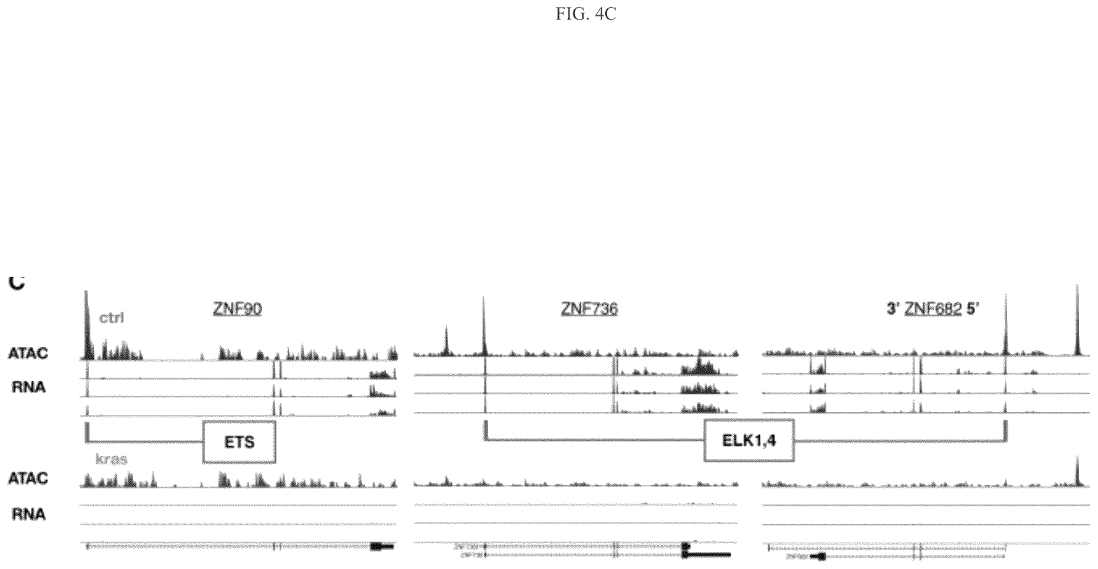

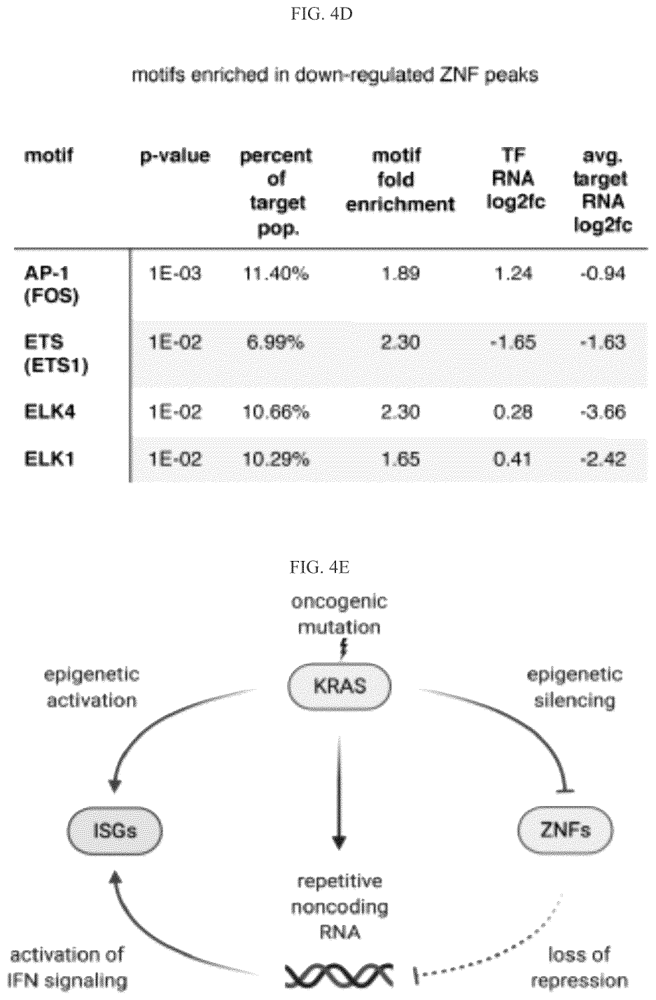

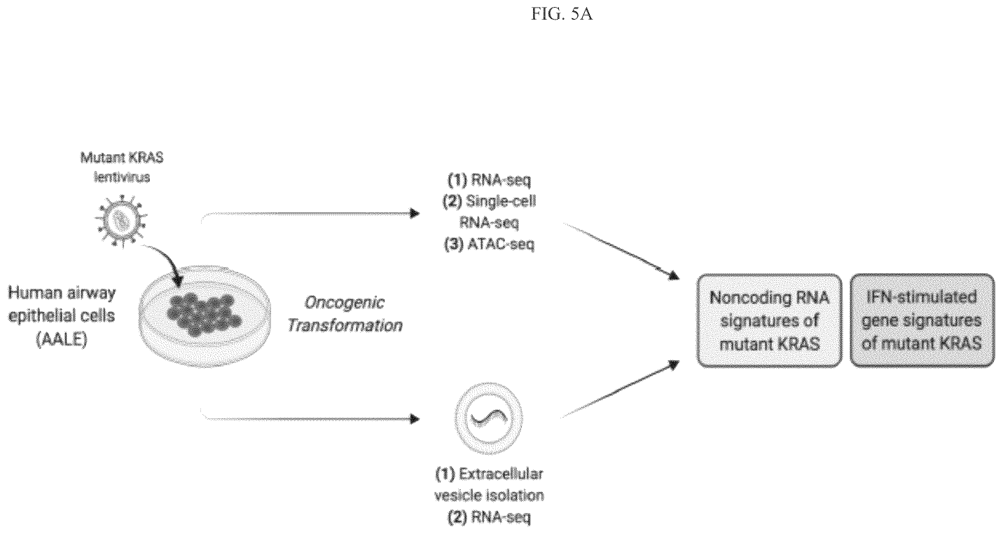

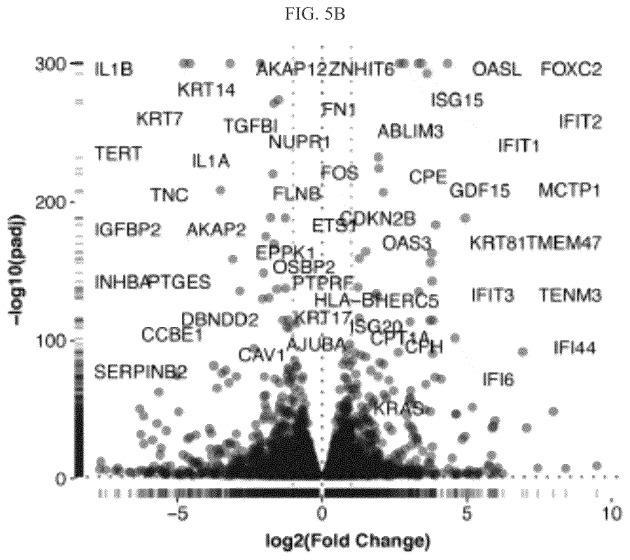

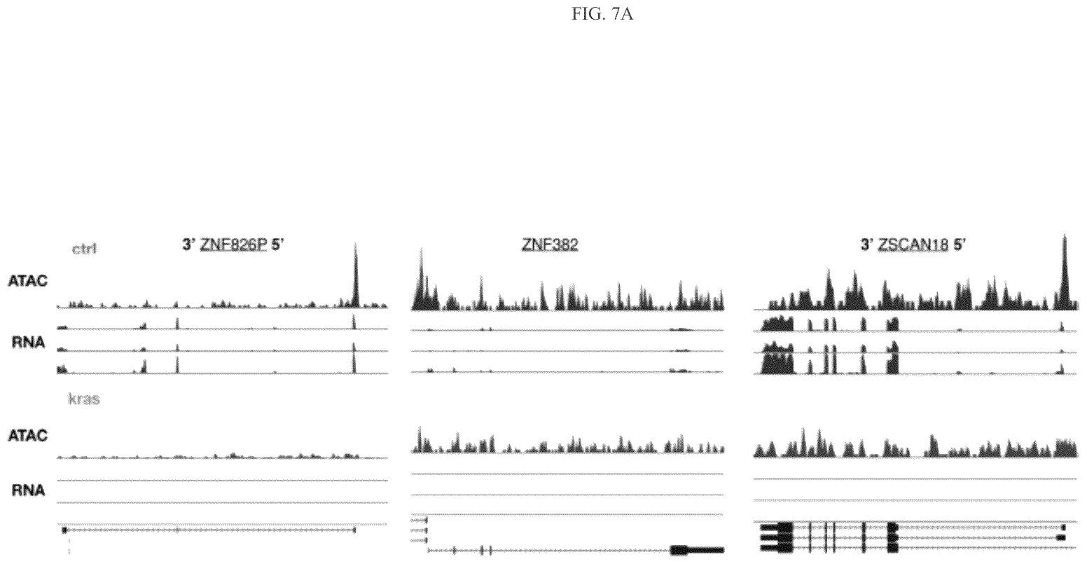

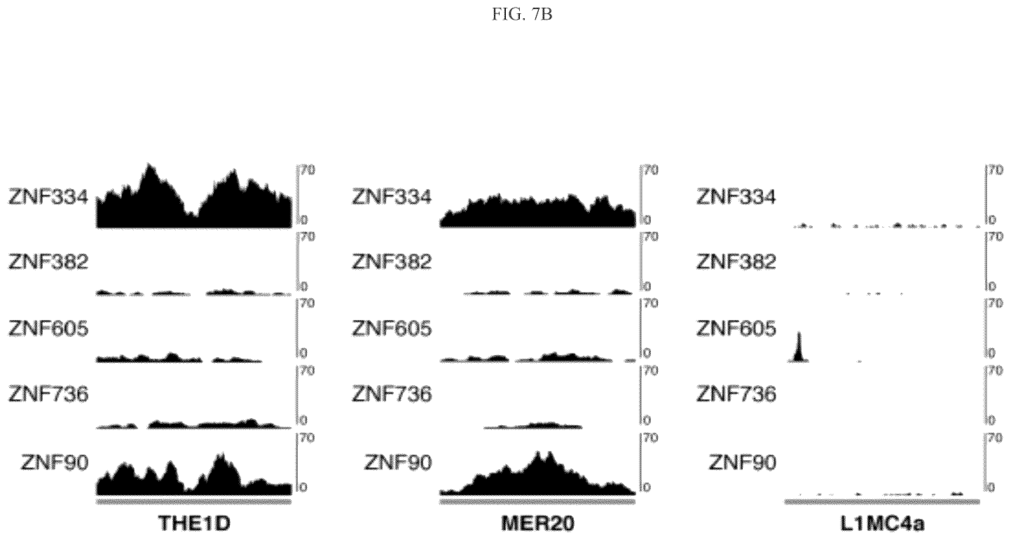

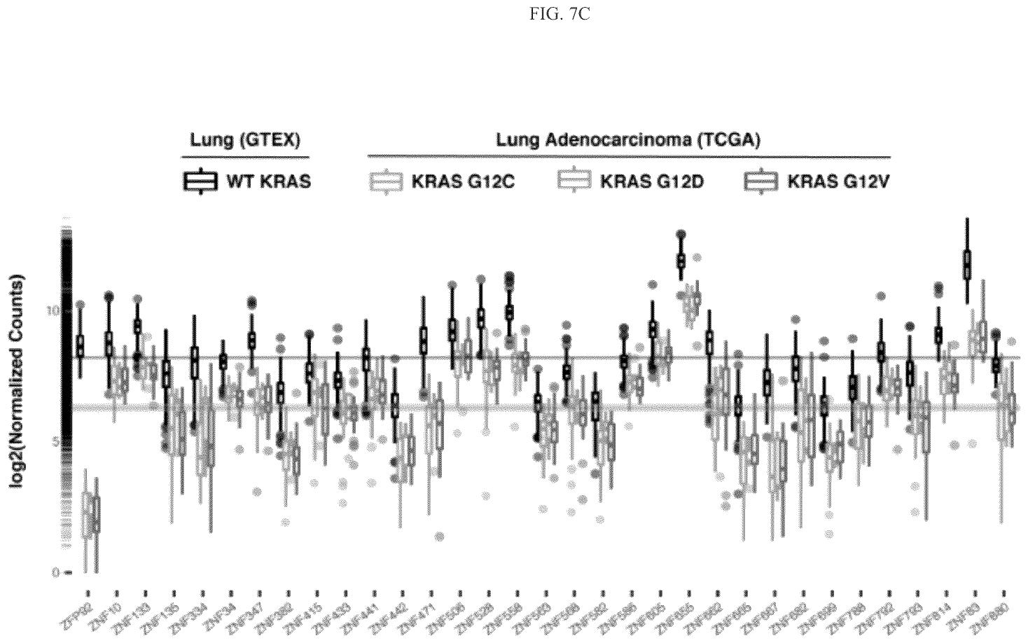

Given the known roles of KRAB zinc-finger proteins (KZNFs) in TE silencing (24), the involvement of KZNFs in TE regulation in mutant KRAS AALEs was examined. KZNFs in mutant KRAS AALEs, showed a broad and significant down-regulation of repressive KRAB domain-containing zinc finger proteins (FIG. 4A) (FIG. 7A). The ATAC-seq experiments indicated that the significantly down-regulated KZNF genes exhibited loss of open chromatin at their TSS and intronic regions (FIG. 4B), revealing that mutant KRAS signaling induces epigenetic silencing of many KZNF genes. Several significantly downregulated KZNFs, including ZNF90, ZNF736, and ZNF683, were enriched for motifs in their TSS regions for ETS and ELK transcription factors (FIG. 4C), which are known downstream effectors of the RAS signaling pathway together with AP-1 (FOS) (11) (FIG. 4D).

KZNF chromatin immunoprecipitation sequencing (ChIP-seq) data (24) was analyzed using the University of California Santa Cruz (UCSC) Repeat Browser platform (25). Several of the significantly down-regulated KZNFs in mutant KRAS AALEs bind to the consensus TE sequences of MER20 and L1MC4a elements (FIG. 7B), which are specifically and significantly unregulated in mutant KRAS AALEs (FIG. 3A). This suggests that suppression of these KZNFs via oncogenic RAS signaling leads to de-repression of TE-derived noncoding RNAs during cellular transformation. This model is supported by broad and significant down-regulation of these same KNZFs in vivo in mutant KRAS-driven lung adenocarcinomas (FIG. 7C).

Collectively, our findings reveal the genomic impact of oncogenic KRAS signaling on repetitive noncoding RNAs and ISGs. Our conclusions are based on deeply sequencing and analyzing the transcriptomes of mutant KRAS-transformed AALE cells at the population, single-cell, and extracellular levels, as well as the epigenomic level, building on work identifying noncoding RNAs that are coordinately regulated with RAS signaling genes during epigenomic reprogramming (7). The molecular basis for the intrinsic ISG signature was observed in mutant KRAS AALE cells is different from TE-induced IFN responses in cancer cells treated with DNA methyltransferase inhibitors (19, 20). Instead the results indicate a prominent role for broad KZNF suppression during early stages of mutant KRAS-driven cellular transformation. The Examples herein suggest oncogenic RAS signaling contributes to the early induction of intrinsic ISG signatures that are observed across many cancers and cancer cells lines with ADAR dependencies (16, 26).

In summary, mutant KRAS both directly and indirectly activates repetitive noncoding RNAs through activation of RAS pathway transcription factors and repression of KZNFs that target these TE-containing loci in the human genome. Moreover, mutant KRAS reprograms the epigenome to both directly and indirectly activate intrinsic ISG signatures through opening chromatin at ISG promoters and activating repetitive noncoding RNAs that are recognized by dsRNA-binding RNA sensors such as PKR and MDA5 (FIG. 4E). Notably, the enrichment of both lncRNAs and TE-derived repetitive noncoding RNAs in extracellular vesicles released from mutant KRAS cells highlights their potential utility as RNA-based liquid biopsy biomarkers for diagnosing RAS-driven cancers.

One or more features from any embodiments described herein or in the figures may be combined with one or more features of any other embodiment described herein in the figures without departing from the scope of the disclosure.

All publications, patents and patent applications cited in this specification are herein incorporated by reference as if each individual publication or patent application were specifically and individually indicated to be incorporated by reference. Although the foregoing disclosure has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

### REFERENCES

1. S. Djebali et al., Landscape of transcription in human cells. Nature 489, 101-108 (2012).
2. E. S. Lander et al., Initial sequencing and analysis of the human genome. Nature 409, 860-921 (2001).
3. K. H. Burns, Transposable elements in cancer. Nat Rev Cancer 17, 415-424 (2017).
4. J. L. Rinn, H. Y. Chang, Long Noncoding RNAs: Molecular Modalities to Organismal Functions. Annu Rev Biochem 89, 283-308 (2020).
5. D. Kelley, J. Rinn, Transposable elements reveal a stem cell-specific class of long noncoding RNAs. Genome Biol 13, R107 (2012).
6. F. J. Slack, A. M. Chinnaiyan, The Role of Non-coding RNAs in Oncology. Cell 179, 1033-1055 (2019).
7. D. H. Kim et al., Single-cell transcriptome analysis reveals dynamic changes in lncRNA expression during reprogramming. Cell Stem Cell 16, 88-101 (2015).
8. O. A. Kent et al., Repression of the miR-143/145 cluster by oncogenic Ras initiates a tumor-promoting feed-forward pathway. Genes Dev 24, 2754-2759 (2010).
9. N. Cancer Genome Atlas Research, Comprehensive molecular profiling of lung adenocarcinoma. Nature 511, 543-550 (2014).
10. E. L. Jackson et al., Analysis of lung tumor initiation and progression using conditional expression of oncogenic K-ras. Genes Dev 15, 3243-3248 (2001).
11. D. K. Simanshu, D. V. Nissley, F. McCormick, RAS Proteins and Their Regulators in Human Disease. Cell 170, 17-33 (2017).
12. a. a. d. h. e. Cancer Genome Atlas Research Network. Electronic address, N. Cancer Genome Atlas Research, Integrated Genomic Characterization of Pancreatic Ductal Adenocarcinoma. Cancer Cell 32, 185-203 e113 (2017).
13. Y. Liu et al., Comparative Molecular Analysis of Gastrointestinal Adenocarcinomas. Cancer Cell 33, 721-735 e728 (2018).
14. A. S. Lundberg et al., Immortalization and transformation of primary human airway epithelial cells by gene transfer. Oncogene 21, 4577-4586 (2002).
15. R. K. Powers, A. Goodspeed, H. Pielke-Lombardo, A. C. Tan, J. C. Costello, GSEA-InContext: identifying novel and common patterns in expression experiments. Bioinformatics 34, i555-i564 (2018).
16. H. Liu et al., Tumor-derived IFN triggers chronic pathway agonism and sensitivity to ADAR loss. Nat Med 25, 95-102 (2019).
17. E. Kim et al., Systematic Functional Interrogation of Rare Cancer Variants Identifies Oncogenic Alleles. Cancer Discov 6, 714-726 (2016).
18. J. D. Buenrostro, B. Wu, H. Y. Chang, W. J. Greenleaf, ATAC-seq: A Method for Assaying Chromatin Accessibility Genome-Wide. Curr Protoc Mol Biol 109, 21 29 21-29 (2015).
19. K. B. Chiappinelli et al., Inhibiting DNA Methylation Causes an Interferon Response in Cancer via dsRNA Including Endogenous Retroviruses. Cell 162, 974-986 (2015).
20. D. Roulois et al., DNA-Demethylating Agents Target Colorectal Cancer Cells by Inducing Viral Mimicry by Endogenous Transcripts. Cell 162, 961-973 (2015).
21. S. H. Roth, E. Y. Levanon, E. Eisenberg, Genome-wide quantification of ADAR adenosine-to-inosine RNA editing activity. Nat Methods 16, 1131-1138 (2019).
22. D. Enderle et al., Characterization of RNA from Exosomes and Other Extracellular Vesicles Isolated by a Novel Spin Column-Based Method. PLoS One 10, e0136133 (2015).
23. F. Liu et al., The Exosome Total Isolation Chip. ACS Nano 11, 10712-10723 (2017).
24. M. Imbeault, P. Y. Helleboid, D. Trono, KRAB zinc-finger proteins contribute to the evolution of gene regulatory networks. Nature 543, 550-554 (2017).
25. J. D. Fernandes et al., The UCSC repeat browser allows discovery and visualization of evolutionary conflict across repeat families. Mob DNA 11, 13 (2020).
26. H. S. Gannon et al., Identification of ADAR1 adenosine deaminase dependency in a subset of cancer cells. Nat Commun 9, 5450 (2018).
27. A. M. Bolger, M. Lohse, B. Usadel, Trimmomatic: a flexible trimmer for Illumina sequence data. Bioinformatics 30, 2114-2120 (2014).
28. J. Brown, M. Pirrung, L. A. McCue, FQC Dashboard: integrates FastQC results into a web-based, interactive, and extensible FASTQ quality control tool. Bioinformatics, (2017).
29. R. Patro, G. Duggal, M. I. Love, R. A. Irizarry, C. Kingsford, Salmon provides fast and bias-aware quantification of transcript expression. Nat Methods 14, 417-419 (2017).
30. A. Dobin et al., STAR: ultrafast universal RNA-seq aligner. Bioinformatics 29, 15-21 (2013).
31. H. Li et al., The Sequence Alignment/Map format and SAMtools. Bioinformatics 25, 2078-2079 (2009).
32. H. Pimentel, N. L. Bray, S. Puente, P. Melsted, L. Pachter, Differential analysis of RNA-seq incorporating quantification uncertainty. Nat Methods 14, 687-690 (2017).
33. C. Soneson, M. I. Love, M. D. Robinson, Differential analyses for RNA-seq: transcript-level estimates improve gene-level inferences. F1000Res 4, 1521 (2015).
34. M. I. Love, W. Huber, S. Anders, Moderated estimation of fold change and dispersion for RNA-seq data with DESeq2. Genome Biol 15, 550 (2014).
35. C. Guo et al., Tau Activates Transposable Elements in Alzheimer's Disease. Cell Rep 23, 2874-2880 (2018).
36. S. Tempel, Using and understanding RepeatMasker. Methods Mol Biol 859, 29-51 (2012).
37. T. Stuart et al., Comprehensive Integration of Single-Cell Data. Cell 177, 1888-1902 e1821 (2019).
38. S. Heinz et al., Simple combinations of lineage-determining transcription factors prime cis-regulatory elements required for macrophage and B cell identities. Mol Cell 38, 576-589 (2010).

What is claimed:

1. A method for detecting a RAS pathway mutation in a subject, the method comprising:
(a) obtaining a biological sample from the subject;
(b) isolating nucleic acids from the biological sample; and
(c) analyzing the expression level of noncoding RNAs in the nucleic acids in conjunction with a corresponding reference level in a control sample,
wherein a differential expression level of the noncoding RNAs compared to the corresponding reference level in the control sample indicates that the subject has a RAS pathway mutation.

2. The method of claim 1 where the RAS pathway mutation is in KRAS, NRAS, HRAS, EGFR, NF1, MET or BRAF.

3. The method of claim 1, wherein the biological sample comprises extracellular vesicles isolated from biofluids from the subject.

4. The method of claim 1, wherein the nucleic acids comprise polyadenylated RNAs.

5. The method of claim 1, wherein the subject has or is suspected of having cancer.

6. The method of claim 5, wherein the cancer is a RAS mutant cancer.

7. The method of claim 6, wherein the RAS mutant cancer is a lung cancer, pancreatic cancer, colorectal cancer, or melanoma.

8. The method of claim 1, wherein the method further comprises analyzing the expression level of a gene involved in the interferon (IFN) alpha, IFN gamma, or KRAB-Zn Finger response.

9. The method of claim 6, wherein an increase in the expression level of the gene involved in the IFN alpha, IFN-gamma, or a decrease in the expression level of a gene involved in the KRAB-Zn finger response relative to a corresponding reference level for the gene in the control sample from the control subject indicates that the subject has cancer.

10. The method of claim 1, wherein the method comprises analyzing the expression level of noncoding RNAs transcribed from transposable elements.

11. The method of claim 10, wherein the transposable element selected from the group consisting of LINE-1 element, LINE-2 element, ERVK element, ERV1 element, EVRL element, EVRL-MaLR element, Alu element, hAT-Charlie element, MIR element, and combinations thereof.

12. The method of claim 11, wherein the LINE-1 element is L1MC4a.

13. The method of claim 11, wherein the Alu element is selected from the group consisting of AluXs, AluSg, AluJo, AluY, AluSz6, and combinations thereof.

14. The method of claim 11, wherein the hAT-Charlie element is MER20.

15. The method of claim 11, wherein the transposable element is selected from the group consisting of AluSx3, MLT1D, AluSz6, L1M1, AluYj4, L1MEc, AluSq, FRAM, AluSz, AluSc, L1HS, AluYc, AluY, AluYa5, AluSg4, AluSx, AluSq2, AluJo, (GAATG)n, AluSc8, AluY, L2a, L1M5, AluYe5, AluSxl, MER11B, AluJr, AluY, AluSc, AluSc8, MSTB2, AluJr, AluYe5, AluSz, L2b, FAM, AluYc, (CATTC)n, AluSc8, Tigger17a, AluY, LOR1b, FRAM, Alu, AluYc, AluY, AluSc8, L1M5, AluSp, AluYh3a3, AluSz6, AluYb8, AluY, AluYf1, AluYa5, MLT1K, L2, L1MA4A, L1MC5, MIRb, AluYb8, AluJo, AluYg6, MLT1B, LTR27C, AluSc8, AluSz6, Charlie3, Tiggerl6a, AluJb, MER4A1, L1MB7, AluSx4, AluSg, L1MC4, L2c, L1M4c, AluYk3, MER1B, AluSq, MIR, LTR16, MamRep38, L1ME4b, AluJb, AluYj4, AluSz, MLT2A1, AluYa5, Tigger19b, L1MC5, AluYc3, AluSg, AluYg6, L1HS, AluYb9, MLT1J1, LTR16B1, L1PA16, MER41A, L1MD2, MIRc, AluSg, L1PA11, LTR16B2, AluSg7, L1PA13, Tigger3b, Tiggerl, AluYa8, AluYh3a3, AluYg6, LTRS, HERVIP10FH-int.

16. The method of claim 1, wherein analyzing the expression level of the noncoding RNAs comprises performing polymerase chain reaction (PCR), reverse transcriptase polymerase chain reaction (RT-PCR), single-cell RNA-sequencing, microarray analysis, a Northern blot, serial analysis of gene expression (SAGE), immunoassay, hybridization capture, cDNA sequencing, direct RNA sequencing, nanopore sequencing, mass spectrometry, a CRISPR based technology, or combinations thereof.

17. The method of claim 1, wherein measuring the expression level of the noncoding RNAs comprises performing sequencing.

18. The method of claim 17, wherein the sequencing comprises obtaining one or more sequencing reads of the noncoding RNAs.

19. The method of claim 18, wherein the analyzing comprises aligning the sequencing reads of the noncoding RNAs to repetitive sequences in a human genome.

20. The method of claim 1, wherein the biological sample is a blood sample, a urine sample, a saliva sample or a tissue sample.

21. The method of claim 20, wherein the biological sample is a tissue sample.

22. The method of claim 1, wherein the subject is a mammal.

23. The method of claim 22, wherein the mammal is a human.

24. The method of claim 1, further comprising administering to the subject one or more anticancer agents.

25. The method of claim 24, wherein the anticaner agent is an inhibitor of a RAS pathway gene.

26. The method of claim 25, wherein the anticancer agent is an inhibitor of KRAS.

27. The method of claim 1, further comprising isolating extracellular vesicles from the sample.

28. The method of claim 27, further comprising isolating extracellular vesicles on the basis of size.

29. The method of claim 28, comprising isolating extracellular vesicles of about 150 nm or about 213 nm in diameter.

30. The method of claim 27, comprising excluding extracellular vesicles of about 196 nm from the sample.
